(19) United States
(12) Patent Application Publication
(10) Pub. No.: US 2005/0176070 A1
(43) Pub. Date: Aug. 11, 2005

(54) Protein analysis

(76) Inventor: Kevin Andrew Auton, Godmanchester (GB)

Correspondence Address: WILSON SONSINI GOODRICH & ROSATI, 650 PAGE MILL ROAD, PALO ALTO, CA, 943041050, US

(21) Appl. No.: 10/473,882

(22) PCT Filed: Apr. 4, 2002
(86) PCT No.: PCT/GB02/01623

(30) Foreign Application Priority Data
Apr. 5, 2001 (GB) 0108521.6
Dec. 28, 2001 (GB) 0131025.9
Feb. 14, 2002 (GB) 0203448.6

(51) Int. Cl. G01N 33/53; C12P 21/06; B05D 3/00

(52) U.S. Cl. 435/7.5; 435/68.1; 427/2.11

(57) ABSTRACT

A method of forming an array of proteins selected from antigens or antibodies; said method comprising the steps of (i) expressing in a recombinant cell, a fusion protein which comprises either (a) an antigen or (b) an antibody binding protein, fused to a peptide having up to 50 amino acids, which peptide comprises amino acid sequence of SEQ ID NO 1 LX₁X₂IX₃X₄X₅X₆KX₇X₈X₉X₁₀ (SEQ ID NO 1) where X₁ is a naturally occurring amino acid, X₂ is any naturally occurring amino acid other than leucine, valine, isoleucine, tryptophan, phenylalanine or tyrosine, X₃ is phenylalanine or leucine, X₄ is glutamine or asparagine, X₅ is alanine, glycine, serine or threonine, X₆ is glycine or methionine, X₇ is isoleucine, methionine or valine, X₈ is glutamine, leucine, valine, tyrosine or isoleucine, X₉ is tryptophan, tyrosine, valine, phenylalanine, leucine or isoleucine and X₁₀ is any naturally occurring amino acid other than asparagine or glutamine; where said peptide is capable of being biotinylated by a biotin ligase at the lysine residue adjacent to X₆; (ii) biotinylating said peptide of the fusion protein at the lysine residue adjacent X₆; (iii) isolating the biotinylated fusion protein; (iv) applying the biotinylated fusion protein to an avidin or streptavidin coated non-porous support; (v) forming an array of at least three different proteins on the support by either (a) where the fusion protein comprises an antigen, carrying out steps (i) to (iv) the desired number of times to form an antigen array; or (b) where the fusion protein comprises an antibody binding protein, applying to said protein, either prior to or after step (iv) a plurality of different antibodies or binding fragments thereof.

### FIELD OF THE INVENTION

The present invention relates to a method of producing arrays for conducting protein analysis, in particular of antibodies, antigens or antibody binding proteins, to protein arrays produced, methods of conducting analysis using them and novel entities incorporated in them. More specifically, the process relates to a method of producing a range of antibodies and/or antigens and immobilising these in an array, for use in protein or binding analysis.

### BACKGROUND

The concept of attaching a number of different proteins to surface supports to form an “array” of proteins has been widely described in the literature (see for example EP0063810, WO84/03151, U.S. Pat. No. 5,143,854).

Recently, there has been a growing interest in the concept of manufacturing devices whereby large numbers of proteins of various classes are arrayed onto different types of solid supports. Examples include antigen, antibody, protein (protein-protein interaction) and functional enzymes arrays.

The background to the technology, and the potential uses for such devices, are thoroughly catalogued in the literature (Joos et al Electrophoresis 2000, 21, 2641-2650, Haab et al Genome Biology 2000 1(6), Borrebaeck Immunology Today, August 2000) and examples of potential utility can be found in a number of recent patent applications including WO 00/07024, WO 99/40434, WO 99/39210 and WO00/54046.

The concept of creating antigen arrays was described in EP 0063810 in 1982. It was reported that antigens and antibodies could be bound to a porous solid support enabling an unlimited number of antibody-antigen interactions to be conducted simultaneously. To make antigen arrays, antigens were simply aliquoted in very small volumes onto nitrocellulose membranes or similar supports, allowed to adsorb and then probed with the corresponding antibodies. As with Enzyme Linked Immunosorbent Assay (ELISA) protocols performed in solution or in plastic plates, non-specific interactions were blocked with Bovine Serum Albumin (BSA), and this is now standard practise. It was also reported that the elements (or spots) of the array did not diffuse and were adsorbed tightly onto the membrane.

It should be noted, however, that the dimensions for these elements were considerably larger than those obtained in micro array device systems. EP 0063810 describes how the protein arrays could be made by aliquoting proteins by hand, using mechanical procedures including a “charged drop” or lithographic process. In this manner elements with a diameter of less than 500 microns (compared with 100 microns that can be achieved with current automated array systems) were produced.

However, one of the main disadvantages associated with the use of membranes as opposed to non-porous surfaces is that the elements tend to diffuse through the support material unless there is immediate binding.

Attempts were made to overcome this problem. U.S. Pat. No. 4,496,654 describes use of porous surfaces such as paper disks which were treated with streptavidin (which is adsorbed onto the surface) enabling arrays of biotinylated antibodies to be arranged in any desired pattern. Following blocking with BSA, the paper discs could be probed with the antigen (exemplified with human chorionic gonadotropin) which could then be detected with an enzyme assay. The biotinylated antibody immediately bound very tightly to the surface of the paper reducing diffusion of the spots.

To achieve this using an “acceptor” surface such as an avidin or streptavidin coated surface, requires that each antibody and antigen, which is attached to the array, must be biotinylated prior to attachment to the array with no guarantee that this process will not impair its avidity (or antigenicity if an antigen is used) compared with the native protein.

Non-porous surfaces also have the disadvantage that they are not as robust as solid surfaces, including various types of glass or plastics, and so cannot be washed or treated as stringently.

For antigen and antibody arrays, it has been found however that attaching a protein to a solid surface generally leads to a reduction in antigenicity of the antigen and avidity of the antibody compared with that observed when the antigen or antibody is in free solution.

Previous attempts (see WO84/03 151 and Haab et al 2000 supra.) to immobilise antigens and antibodies were not greatly successful. WO84/03 151 describes that antibodies can be applied directly onto glass surfaces such as a microscope cover slip and dried. When blocked and then exposed to antigens, in this case in the form of whole cells, the antigens were captured by the array. However, WO84/03 151 further describes that these antigens needed to be added at a higher concentration compared with the equivalent ELISA performed in solution. It was also noted that the antibodies had to be “highly enriched in order to achieve a sufficiently dense antibody coat for the desired cell adherence”. It also took considerable time for the antibodies to be adsorbed onto the glass surface.

Other approaches for the direct immobilisation of antigens and antibodies have been reported. One approach was to first adsorb calcium phosphate in the form of hydroxyapatite onto filter papers onto which proteins were bound by ionic interaction as described in U.S. Pat. No. 5,827,669. These inventors reported that this was not effective for acidic proteins and that the antibodies suffered from “bad orientation” onto the Ca/phosphate layer. Success was, however, reported when this method was used in immobilising streptavidin.

Another method for immobilising proteins to solid, non-porous surfaces included attaching them using an adhesive polyphenolic protein isolated from muscles as described in U.S. Pat. No. 5,817,470. By coating solid surfaces, such as a polystyrene multi-well plate with polyphenolic protein, various antigens could be bound to the treated support and detected in an ELISA sandwich comprising of a primary antibody followed by a secondary antibody conjugated to an enzyme.

However, the inventors conceded that the procedure was limited by the amount of antigen bound or adsorbed to the solid surface. The final amount of antigen strongly bound to the surface of the plate varied depending on a number of factors such as the molecular characteristics of the antigens, the properties of the solid support, the concentration of the antigen in the solution as well as the characteristics of the buffer used to dissolve the antigen used to coat or to activate the surface. In general, only a small fraction of the antigen present in the coating solution was adsorbed to the surface.

Direct attachment of antibodies and antigens to non-porous surfaces was also been attempted with a collection of 113 antibodies and their corresponding antigens (Haab et al, 2000 Genome Biology 1(6)). By exploiting technology developed for DNA microarrays, glass slides coated with poly-s-lysine were used to immobilise both antigens in one experiment and antibodies in another. The results reported showed that only 50% of the arrayed antigens and 20% of the arrayed antibodies, provided specific and accurate measurements of their cognate ligands at or below concentrations of 1.60 μl/ml and 0.34 μg/ml respectively.

The high failure rate in binding antibodies to solid surfaces would not be acceptable for a large-scale antibody array manufacturing programme. This supports the view that direct attachment of antigens and antibodies is an unsuitable technique to retain antibody/antigen functionality if protein arrays are to fulfil their potential.

The use of coatings such as avidin and streptavidin as binders for biotin labelled proteins is well known for use in conjunction with many proteins. The proteins are generally isolated first, and then biotinylated. Biotin can be conjugated to the protein at any or all active lysine sites contained within it.

Thus, when antigens or antibodies are biotinylated in this way, biotin groups may be present at their N-terminal groups and at any number of potential active lysine residues over their surface. This means that they will adopt any number of different orientations once bound to the streptavidin layer and so the binding properties will be diverse. Furthermore, access to the antigen or antibody immobilised via streptavidin will be reduced by steric hindrance, leading to generally inadequate assay.

It has been found that it is possible to reduce the steric hindrance and increase the sensitivity of the immunoassay by including a linker between the antigen/antibody and the biotinylated site.

U.S. Pat. No. 5,811,246 describes how small synthetic peptides used in either immunoassays or for raising antisera can be linked to a “carrier” protein such as avidin or streptavidin via a linker such as various bradykinin derivatives. This has several advantages. Firstly, the condensation reaction between the free N-terminal group on the peptide and the linker preserves the charged residues essential for recognition by an antibody (immunoassay) or to elicit an immune response (immunisation). Secondly, the bradykinin linker can then be biotinylated in such a way as to preserve the free charged groups on the small peptide. In each case, the presence of the linker appears to promote a more sensitive immunoassay and an improved immune response when used as an immunising agent.

This use of a bradykinin derivative in this way however introduces further steps and complications into the process.

Biotinylated peptides fused to peptides or proteins of interest are described in U.S. Pat. Nos. 5,723,584, 5,874,239 and 5,932,433, and further in Beckett et a!. Protein Sci. (1999) 8(4) 921-9. These peptides are used in order to biotinylate recombinant proteins, so as to allow rapid purification, immobilization, labelling and detection thereof. It is not suggested that these peptides should be used in particular with antigens or antibody binding proteins, or that they should be formulated in arrays.

The present applicants have found that the peptides used in these patents allow the production of very good antigen or antibody arrays, which can be efficiently produced on non-porous supports whilst substantially retaining the binding avidity of these proteins.

### SUMMARY OF THE INVENTION

According to a first aspect of the present invention there is provided a method of forming an array of proteins selected from antigens or antibodies; said method comprising the steps of

(i) expressing in a recombinant cell, a fusion protein which comprises either (a) an antigen or (b) an antibody binding protein, fused to a peptide having up to 50 amino acids, which peptide comprised amino acid sequence of SEQ ID NO 1

| | |
|---|---|
| LX₁X₂IX₃X₄X₅X₆KX₇X₈X₉X₁₀ | (SEQ ID NO 1) |

where X1 is a naturally occurring amino acid, X₂ is any naturally occurring amino acid other than leucine, valine, isoleucine, tryptophan, phenylalanine or tyrosine, X₃ is phenylalanine or leucine, X₄ is glutamine or asparagine, X₅ is alanine, glycine, serine or threonine, X₆ is glycine or methionine, X₇ is isoleucine, methionine or valine, X₈ is glutamine, leucine, valine, tyrosine or isoleucine, X₉ is tryptophan, tyrosine, valine, phenylalanine, leucine or isoleucine and X₁₀ is any naturally occurring amino acid other than asparagine or glutamine; where said peptide is capable of being biotinylated by a biotin ligase at the lysine residue adjacent to X₆;
(ii) biotinylating said peptide of the fusion protein at the lysine residue adjacent X₆;
(iii) isolating the biotinylated fusion protein;
(iv) applying the biotinylated fusion protein to an avidin or streptavidin coated non-porous support;
(v) forming an array of at least three different proteins on the support by either
(a) where the fusion protein comprises an antigen, carrying out steps (i) to (iv) the desired number of times to form an antigen array; or
(b) where the fusion protein comprises an antibody binding protein, applying to said protein, either prior to or after step (iv) a plurality of different antibodies or binding fragments thereof.

The applicants have found that by using a fusion of the antigen or antibody binding protein to a peptide of SEQ ID NO 1, these proteins may be immobilised onto solid surfaces, whilst substantially maintaining the antigenicity of proteins, or the binding capabilities of the antibody binding proteins.

This may be because the fusion peptide is biotinylated rather than the protein itself, and so there is less disruption of the protein's antigenicity when attached to the support surface. In addition, the peptide including SEQ ID NO 1 appears to reduce steric hindrance to enable interaction between antigen and antibody. By ensuring that the peptide linker is attached at a terminal region of the protein, and contains the biotinylation site, sites on the protein which are essential for function appear to be largely unaffected. This combination is particularly advantageous in the context of methods of analysis using antigens or antibody arrays.

The method described herein represents the first time that the mode of attachment of proteins to non-porous surfaces (step vi), the mode of protein isolation from cell lysate (step iii) and the method of biotinylation (step ii) utilise the same fusion peptide.

Unless defined otherwise, all technical and scientific terms used herein have the same meaning as commonly understood by one of ordinary skill in the art.

As used herein the expression “antibody binding protein” refers to proteins which are known to bind to regions of antibodies, or to mixtures of these. Examples of such proteins include Protein A, Protein L and Protein G

Antibody binding proteins are used in accordance with the invention in the production of antibody arrays. The antibodies are bound by antibody binding proteins, such as Proteins A, G and/or L or a mixture of one or more of these, which are themselves anchored via the linker to the streptavidin coating on the support surface. While biotinylated versions of native Protein A, G and L are commercially available and can be attached to the streptavidin coating on the support surface, the applicants have found that by fusing these proteins to biotinylated tags in accordance with the present invention at the C and/or N-terminals, highly effective binding of antibodies of various types was achieved. This may also be the result of reduced steric factors, or that the binding sites on the proteins are all readily available.

In addition, by using the method of the invention, the biotinylated fusion protein is immediately captured on application to the avidin or streptavidin coated support in step (iv) leading to very discrete spots of protein on the support, with minimal observable diffusion.

Particular examples of peptides having up to 50 amino acids, which peptide comprises an amino acid sequence of SEQ ID NO 1 are listed in U.S. Pat. No. 5,723,584, U.S. Pat. No. 5,874,239 and U.S. Pat. No. 5,932,433, the content of which are incorporated herein by reference. Examples of peptides provided in these references are listed below:

| | |
|---|---|
| Leu Glu Glu Val Asp Ser Thr Ser | (SEQ ID NO: 14) |
| Ser Ala Ile Phe Asp Ala Met Lys | |
| Met Val Trp Ile Ser Pro Thr Glu | |
| Phe Arg; | |
| Gln Gly Asp Arg Asp Glu Thr Leu | (SEQ ID NO: 15) |
| Pro Met Ile Leu Arg Ala Met Lys | |
| Met Glu Val Tyr Asn Pro Gly Gly | |
| His Glu Lys; | |
| Ser Lys Cys Ser Tyr Ser His Asp | (SEQ ID NO: 16) |
| Leu Lys Ile Phe Glu Ala Gln Lys | |
| Met Leu Val His Ser Tyr Leu Arg | |
| Val Met Tyr Asn Tyr; | |
| Met Ala Ser Ser Asp Asp Gly Leu | (SEQ ID NO: 17) |
| Leu Thr Ile Phe Asp Ala Thr Lys | |
| Met Met Phe Ile Arg Thr; | |
| Ser Tyr Met Asp Arg Thr Asp Val | (SEQ ID NO: 18) |
| Pro Thr Ile Leu Glu Ala Met Lys | |
| Met Glu Leu His Thr Thr Pro Trp | |
| Ala Cys Arg; | |
| Ser Phe Pro Pro Ser Leu Pro Asp | (SEQ ID NO: 19) |
| Lys Asn Ile Phe Glu Ala Met Lys | |
| Met Tyr Val Ile Thr; | |
| Ser Val Val Pro Glu Pro Gly Trp | (SEQ ID NO: 20) |
| Asp Gly Pro Phe Glu Ser Met Lys | |
| Met Val Tyr His Ser Gly Ala Gln | |
| Ser Gly Gln; | |
| Val Arg His Leu Pro Pro Pro Leu | (SEQ ID NO: 21) |
| Pro Ala Leu Phe Asp Ala Met Lys | |
| Met Glu Phe Val Thr Ser Val Gln | |
| Phe; | |
| Asp Met Thr Met Pro Thr Gly Met | (SEQ ID NO: 22) |
| Thr Lys Ile Phe Glu Ala Met Lys | |
| Met Glu Val Ser Thr; | |
| Ala Thr Ala Gly Pro Leu His Glu | (SEQ ID NO: 23) |
| Pro Asp Ile Phe Leu Ala Met Lys | |
| Met Glu Val Val Asp Val Thr Asn | |
| Lys Ala Gly Gln; | |
| Ser Met Trp Glu Thr Leu Asn Ala | (SEQ ID NO: 24) |
| Gln Lys Thr Val Leu Leu; | |
| Ser His Pro Ser Gln Leu Met Thr | (SEQ ID NO: 25) |
| Asn Asp Ile Phe Glu Gly Met Lys | |
| Met Leu Tyr His; | |
| Thr Ser Glu Leu Ser Lys Leu Asp | (SEQ ID NO: 27) |
| Ala Thr Ile Phe Ala Ala Met Lys | |
| Met Gln Trp Trp Asn Pro Gly; | |
| Val Met Glu Thr Gly Leu Asp Leu | (SEQ ID NO: 28) |
| Arg Pro Ile Leu Thr Gly Met Lys | |
| Met Asp Trp Ile Pro Lys; | |
| Leu His His Ile Leu Asp Ala Gln | (SEQ ID NO: 30) |
| Lys Met Val Trp Asn His Arg; | |
| Pro Gln Gly Ile Phe Glu Ala Gln | (SEQ ID NO: 31) |
| Lys Met Leu Trp Arg Ser; | |
| Leu Ala Gly Thr Phe Glu Ala Leu | (SEQ ID NO: 32) |
| Lys Met Ala Trp His Glu His; | |
| Leu Asn Ala Ile Phe Glu Ala Met | (SEQ ID NO: 33) |
| Lys Met Glu Tyr Ser Gly; | |
| Leu Gly Gly Ile Phe Glu Ala Met | (SEQ ID NO: 34) |
| Lys Met Glu Leu Arg Asp; | |
| Leu Leu Arg Thr Phe Glu Ala Met | (SEQ ID NO: 35) |
| Lys Met Asp Trp Arg Asn Gly; | |
| Leu Ser Thr Ile Met Glu Gly Met | (SEQ ID NO: 36) |
| Lys Met Tyr Ile Gln Arg Ser; | |
| Leu Ser Asp Ile Phe Glu Ala Met | (SEQ ID NO: 37) |
| Lys Met Val Tyr Arg Pro Cys; | |
| Leu Glu Ser Met Leu Glu Ala Met | (SEQ ID NO: 38) |
| Lys Met Gln Trp Asn Pro Gln; | |
| Leu Ser Asp Ile Phe Asp Ala Met | (SEQ ID NO: 39) |
| Lys Met Val Tyr Arg Pro Gln; | |
| Leu Ala Pro Phe Phe Glu Ser Met | (SEQ ID NO: 40) |
| Lys Met Val Trp Arg Glu His; | |
| Leu Lys Gly Ile Phe Glu Ala Met | (SEQ ID NO: 41) |
| Lys Met Glu Tyr Thr Ala Met; | |
| Leu Glu Gly Ile Phe Glu Ala Met | (SEQ ID NO: 42) |
| Lys Met Glu Tyr Ser Asn Ser; | |
| Leu Leu Gln Thr Phe Asp Ala Met | (SEQ ID NO: 43) |
| Lys Met Glu Trp Leu Pro Lys; | |
| Val Phe Asp Ile Leu Glu Ala Gln | (SEQ ID NO: 44) |
| Lys Val Val Thr Leu Arg Phe; | |
| Leu Val Ser Met Phe Asp Gly Met | (SEQ ID NO: 45) |
| Lys Met Glu Trp Lys Thr Leu; | |
| Leu Glu Pro Ile Phe Glu Ala Met | (SEQ ID NO: 46) |
| Lys Met Asp Trp Arg Leu Glu; | |
| Leu Lys Glu Ile Phe Glu Gly Met | (SEQ ID NO: 47) |
| Lys Met Glu Phe Val Lys Pro; | |
| Leu Gly Gly Ile Glu Ala Gln Lys | (SEQ ID NO: 48) |
| Met Leu Leu Tyr Arg Gly Asn; | |
| Arg Pro Val Leu Glu Asn Ile Phe | (SEQ ID NO: 50) |
| Glu Ala Met Lys Met Glu Val Trp | |
| Lys Pro; | |
| Arg Ser Pro Ile Ala Glu Ile Phe | (SEQ ID NO: 51) |
| Glu Ala Met Lys Met Glu Tyr Arg | |
| Glu Thr; | |
| Gln Asp Ser Ile Met Pro Ile Phe | (SEQ ID NO: 52) |
| Glu Ala Met Lys Met Ser Trp His | |
| Val Asn; | |
| Asp Gly Val Leu Phe Pro Ile Phe | (SEQ ID NO: 53) |
| Glu Ala Met Lys Met Ile Arg Leu | |
| Glu Thr; | |
| Val Ser Arg Thr Met Thr Asn Phe | (SEQ ID NO: 54) |
| Glu Ala Met Lys Met Ile Tyr His | |
| Asp Leu; | |
| Asp Val Leu Leu Pro Thr Val Phe | (SEQ ID NO: 55) |
| Glu Ala Met Lys Met Tyr Ile Thr | |
| Lys; | |
| Pro Asn Asp Leu Glu Arg Ile Phe | (SEQ ID NO: 56) |
| Asp Ala Met Lys Ile Val Thr Val | |
| His Ser; | |
| Thr Arg Ala Leu Leu Glu Ile Phe | (SEQ ID NO: 57) |
| Asp Ala Gln Lys Met Leu Tyr Gln | |
| His Leu; | |
| Arg Asp Val His Val Gly Ile Phe | (SEQ ID NO: 58) |
| Glu Ala Met Lys Met Tyr Thr Val | |
| Glu Thr; | |
| Gly Asp Lys Leu Thr Glu Ile Phe | (SEQ ID NO: 59) |
| Glu Ala Met Lys Ile Gln Trp Thr | |
| Ser Gly; | |
| Leu Glu Gly Leu Arg Ala Val Phe | (SEQ ID NO: 60) |
| Glu Ser Met Lys Met Glu Leu Ala | |
| Asp Glu; | |
| Val Ala Asp Ser His Asp Thr Phe | (SEQ ID NO: 61) |
| Ala Ala Met Lys Met Val Trp Leu | |
| Asp Thr; | |
| Gly Leu Pro Leu Gln Asp Ile Leu | (SEQ ID NO: 62) |
| Glu Ser Met Lys Ile Val Met Thr | |
| Ser Gly; | |
| Arg Val Pro Leu Glu Ala Ile Phe | (SEQ ID NO: 63) |
| Glu Gly Ala Lys Met Ile Trp Val | |
| Pro Asn Asn; | |
| Pro Met Ile Ser His Lys Asn Phe | (SEQ ID NO: 64) |
| Glu Ala Met Lys Met Lys Phe Val | |
| Pro Glu; | |
| Lys Leu Gly Leu Pro Ala Met Phe | (SEQ ID NO: 65) |
| Glu Ala Met Lys Met Glu Trp His | |
| Pro Ser; | |
| Gln Pro Ser Leu Leu Ser Ile Phe | (SEQ ID NO: 66) |
| Glu Ala Met Lys Met Gln Ala Ser | |
| Leu Met; | |
| Leu Leu Glu Leu Arg Ser Asn Phe | (SEQ ID NO: 67) |
| Glu Ala Met Lys Met Glu Trp Gln | |
| Ile Ser; | |
| Asp Glu Glu Leu Asn Gln Ile Phe | (SEQ ID NO: 68) |
| Glu Ala Met Lys Met Tyr Pro Leu | |
| Val His Val Thr Lys; | |
| Ser Asn Leu Val Ser Leu Leu His | (SEQ ID NO: 70) |
| Ser Gln Lys Ile Leu Trp Thr Asp | |
| Pro Gln Ser Phe Gly; | |
| Leu Phe Leu His Asp Phe Leu Asn | (SEQ ID NO: 71) |
| Ala Gln Lys Val Glu Leu Tyr Pro | |
| Val Thr Ser Ser Gly; | |
| Ser Asp Ile Asn Ala Leu Leu Ser | (SEQ ID NO: 72) |
| Thr Gln Lys Ile Tyr Trp Ala His; | |
| Met Ala Ser Ser Leu Arg Gln Ile | (SEQ ID NO: 73) |
| Leu Asp Ser Gln Lys Met Glu Trp | |
| Arg Ser Asn Ala Gly Gly Ser; | |
| Met Ala His Ser Leu Val Pro Ile | (SEQ ID NO: 75) |
| Phe Asp Ala Gln Lys Ile Glu Trp | |
| Arg Asp Pro Phe Gly Gly Ser; | |
| Met Gly Pro Asp Leu Val Asn Ile | (SEQ ID NO: 76) |
| Phe Glu Ala Gln Lys Ile Glu Trp | |
| His Pro Leu Thr Gly Gly Ser; | |
| Met Ala Phe Ser Leu Arg Ser Ile | (SEQ ID NO: 77) |
| Leu Glu Ala Gln Lys Met Glu Leu | |
| Arg Asn Thr Pro Gly Gly Ser; | |
| Met Ala Gly Gly Leu Asn Asp Ile | (SEQ ID NO: 78) |
| Phe Glu Ala Gln Lys Ile Glu Trp | |
| His Glu Asp Thr Gly Gly Ser; | |
| Met Ser Ser Tyr Leu Ala Pro Ile | (SEQ ID NO: 79) |
| Phe Glu Ala Gln Lys Ile Glu Trp | |
| His Ser Ala Tyr Gly Gly Ser; | |
| Met Ala Lys Ala Leu Gln Lys Ile | (SEQ ID NO: 80) |
| Leu Glu Ala Gln Lys Met Glu Trp | |
| Arg Ser His Pro Gly Gly Ser; | |
| Met Ala Phe Gln Leu Cys Lys Ile | (SEQ ID NO: 81) |
| Phe Tyr Ala Gln Lys Met Glu Trp | |
| His Gly Val Gly Gly Gly Ser; | |
| Met Ala Gly Ser Leu Ser Thr Ile | (SEQ ID NO: 82) |
| Phe Asp Ala Gln Lys Ile Glu Trp | |
| His Val Gly Lys Gly Gly Ser; | |
| Met Ala Gln Gln Leu Pro Asp Ile | (SEQ ID NO: 83) |
| Phe Asp Ala Gln Lys Ile Glu Trp | |
| Arg Ile Ala Gly Gly Gly Ser; | |
| Met Ala Gln Arg Leu Phe His Ile | (SEQ ID NO: 84) |
| Leu Asp Ala Gln Lys Ile Glu Trp | |
| His Gly Pro Lys Gly Gly Ser; | |
| Met Ala Gly Cys Leu Gly Pro Ile | (SEQ ID NO: 85) |
| Phe Glu Ala Gln Lys Met Glu Trp | |
| Arg His Phe Val Gly Gly Ser; | |
| Met Ala Trp Ser Leu Lys Pro Ile | (SEQ ID NO: 86) |
| Phe Asp Ala Gln Lys Ile Glu Trp | |
| His Ser Pro Gly Gly Gly Ser; | |
| Met Ala Leu Gly Leu Thr Arg Ile | (SEQ ID NO: 87) |
| Leu Asp Ala Gln Lys Ile Glu Trp | |
| His Arg Asp Ser Gly Gly Ser; | |
| Met Ala Gly Ser Leu Arg Gln Ile | (SEQ ID NO: 88) |
| Leu Asp Ala Gln Lys Ile Glu Trp | |
| Arg Arg Pro Leu Gly Gly Ser, | |
| and; | |
| Met Ala Asp Arg Leu Ala Tyr Ile | (SEQ ID NO: 89) |
| Leu Glu Ala Gln Lys Met Glu Trp | |
| His Pro His Lys Gly Gly Ser. | |

These peptides, or fragments thereof which include SEQ ID NO 1 are suitable examples of peptides for use in producing fusion proteins in step (i).

In particular, the peptides used in the method of the invention to form the fusion protein have from 13 to 20 amino acids, and preferably about 15 amino acids.

A particularly preferred peptide for use in the fusion protein of the invention is a 15 amino acid peptide fragment of SEQ ID NO 78 shown above. Specifically, a preferred peptide is of amino acid sequence SEQ ID NO 2:

| | |
|---|---|
| Gly Leu Asn Asp Ile Phe Glu Ala Gln | (SEQ ID NO 2) |
| Lys Ile Glu Trp His Glu. | |

This peptide is known as AviTag™ and DNA vectors encoding this sequence are available from Avidity Inc., sold under the trade names pAN4, pAN-5 and pAN-6 (which are suitable for producing fusion proteins in which the peptide of SEQ ID NO 2 is attached at the N terminus of the protein) and pAC-4, pAC-5 and pAC-6 (which are suitable for producing fusion proteins in which the peptide of SEQ ID NO 2 is attached at the C terminus of the protein). The sequence of these vectors are shown hereinafter as SEQ ID NO 3, SEQ ID NO 4, SEQ ID NO 5, SEQ ID NO 6, SEQ ID NO 7 and SEQ ID NO 8 in FIGS. 7-12 respectively. These vectors further include the ampicillin resistance gene bla to assist in cloning. However the AviTag™ sequence can also be transferred into other vector systems.

Biotinylation can be effected in various ways, either in vivo or in vitro, for example by by co-expressing biotin ligase in the expression host, by adding biotin ligase to the cell lysate or by adding the biotin ligase to the purified protein. In a particularly preferred embodiment, the method utilises the ability to enzymatically biotinylate a lysine residue in the fusion peptide in vivo prior to protein isolation from the cell lysate, by co-expressing biotin ligase in the expression host. Usually when in vitro techniques are used, the expressed protein must first be isolated from the cell lysate and then chemically biotinylated in vitro by means well known in the art. This results in loss of material and random biotinylation. Proteins with multiple biotinylated sites have an unpredictable orientation and degree of binding onto the capture surface. The advantage of the method of the invention is that all expressed proteins will be uniformly attached via the same residue on the same linker to the array

Thus, suitably, the recombinant cell used in step (i) of the invention is engineered such that it also expresses a biotinylating enzyme and also contains biotin, such that step (ii) is effected in vivo in said cell as illustrated diagrammatically hereinafter in FIG. 1. DNA (1), which is suitably a cloned gene encoding an antigen or an antibody binding protein is sub-cloned into a vector (2) (such as pAN4, pAN-5, pAN-6 or pAC4, pAC-5 or pAC-6) which includes a sequence (3) encoding a peptide of SEQ ID NO. 1. The subcloned gene is then expressed in an expression system such as E. coli, which has been transformed with the vector as a fusion protein (4) comprising the antigen or antibody binding protein (5) fused to a peptide (6) of SEQ ID NO. 1. When expressed in-vivo in the presence of constitutively expressed biotin ligase, the lysine residue on the fusion peptide (6) is enzymatically biotinylated.

If the cell does not produce biotin, then it may be added to the culture medium in order to produce the desired result. This reduces the number of steps involved in the process.

A particularly suitable host cell for use in the method of the invention are the AVB100, AVB 101 and AVB99 E. coli strains available from Avidity Inc., Denver, Colo., USA. These strains all have the birA gene stably integrated into the chromosome so that they express biotin ligase. In the case of AVB 100, overexpression of of BirA protein may be achieved by induction with L-arabinose. The AVB101 E. coli B strain contains the pACYC184 ColE1 compatible plasmid that over-expresses biotin ligase, the elevated levels of Biotin Ligase in the cells result in complete biotinylation of fusion proteins in vivo. An alternative host cell is strain AVB99 (Avidity Inc) which is an E. coli strain (XL1-Blue) containing a pACYC184 plasmid with an IPTG-inducible birA gene to overexpress biotin ligase (pBirAcm).

Fusion proteins produced in step (i) may also be isolated and biotinylated in vitro in the usual way. The structure of the peptide of SEQ ID NO 1 is such that biotinylation will occur reliably at lysine adjacent X₆ within SEQ ID NO 1.

In a preferred embodiment of the invention, the peptide comprising the amino acid of SEQ ID NO 1 is also used as a means of isolating the fusion protein in step (iii) of the method. The technology for protein expression using recombinant DNA technology is well known in the art. However, each protein that is expressed has a different amino acid sequence and many sequences are either difficult to express in the host of choice or their sequence is hydrophobic, and therefore insoluble, or is toxic to the host. Even in the simplest bacterial expression systems, inclusion bodies are often formed that are difficult to disrupt while leaving the target protein in its native active state.

It is now common practise to fuse the target protein with another protein/peptide sequence (tag) to aid the purification process subsequent to expression. Examples of such fusion expression systems are now widely used and have been commercialised by several suppliers. Such fusion peptide sequences are attached to the amino or carboxyl terminal end of a protein sequence and are recognised by specific antibodies or affinity resins.

The expressed proteins must be solubilised from the cellular debris sometimes requiring harsh conditions including unphysiological pH values or use of chaotropic reagents and therefore the affinity purification process must be robust enough to function under such conditions.

By using this sequence as a means of isolating or purifying the expressed fusion protein, the need for additional purification tags is eliminated. Thus this sequence has a dual purpose.

In some cases it may be desirable to use a further peptide sequence tag as a means of isolating or purifying the expressed fusion protein. The sequence is preferably between 1 and 30 amino acids in length.

The peptide sequence tag sequence (20) may be located at the N-terminal or C-terminal region of the antigen or antibody binding protein as shown in FIG. 13. It is, however, preferably located at the opposite end of the antigen or antibody binding protein to which SEQ ID NO 1 is fused. Where the additional peptide sequence tag is located on the same terminal region as SEQ ID NO 1, it is preferably fused to the free end of SEQ ID NO 1.

Many peptide sequence tags are known in the art. Examples of suitable peptide sequence tags for the purposes of the present invention are described in U.S. Pat. No. 4,569,794A, and EP0 282 042B, the contents of which are herein incorporated by reference.

Preferably, the peptide sequence tag comprises at least one histidine amino acid. Even more preferably the peptide sequence tag has the formula His-X in which X is selected from the group consisting of -Gly-, -His-, -Tyr-, -Gly-, -Trp-, -Val-, -Leu-, -Ser-, -Lys-, -Phe-, -Met-, -Ala-, -Glu-, -Ile-, -Thr-, -Asp-, -Asn-, -Gln-, -Arg-, -Cys- and -Pro-.

Alternatively the peptide sequence tag has the formula Y-His wherein Y is selected from -Gly-, -Ala-, -His- and -Tyr-.

Particularly suitable peptide sequence tags are described in EP 0 282 042B, and a preferred example is a hexa His tag.

In one embodiment of the method of the invention, step (iii) is effected using a further antibody or a binding fragment thereof, which is specific for the peptide of amino acid sequence including SEQ ID NO 1. The said further antibody may be raised using conventional techniques to the peptide (7) which includes an amino acid of SEQ ID NO 1. This method is illustrated diagrammatically in FIG. 2.

The said further antibody is an anti-fusion antibody (8), which may be immobilised on a column, magnetic bead (9) or pipette tip, for example using a secondary antibody which is suitably an anti-species antibody (10) or other methods described in the literature, such as using an antibody binding protein such as Protein A, Protein G or Protein L, bound to the bead (9). This approach is highly suited to automation and to the isolation of large numbers, but small quantities, of novel fusion proteins in parallel. After separation from the cell lysate residue, the bound fusion protein (4) can subsequently be eluted by increasing the pH from 7.0 to 9.0.

In an alternative embodiment, the fusion protein is isolated using a separation material which has some affinity for biotin but which releases the biotin fairly readily. Suitably the separation material is a modified version of avidin or streptavidin, which has lower affinity for biotin than native avidin or streptavidin. A particular example of such a material is a modified version of avidin marketed as CaptAvidin™ by by Molecular Probes (Eugene, Oreg., USA).

In this embodiment, the fusion protein is isolated from the cellular debris, detergents and salts etc from the culture medium, by lowering the pH of the cell lysis mixture to pH 6.0 followed by affinity purification with CaptAvidin™ attached to (a) magnetic beads or (b) pipette tips using conventional methods. Bound fusion protein may then be eluted from the magnetic beads or mini columns by subsequently increasing the pH from 6.0 to 9.5.

Prior to the step (iv), it is preferable to confirm the identity of the expressed fusion protein. In one particular technique, a very low volume (10 μl) of the isolated fusion protein is removed from the microtitre plate. The sample is digested by trypsin (using methods well known in the art). The resultant peptide extract is desalted and concentrated using a ZipTip™ (Millipore, Mass., USA) or equivalent, before analysis via mass spectrometry. Since the sequence of the fusion protein is known, identification by MALDI spectrometry to identify the peptides is usually sufficient to confirm the identity of the fusion protein. This technique is widely used in protein research and is summarised by T. Rabilloud (Editor) Proteome Research: 2D gel electrophoresis and identification methods. Furthermore, this technique can be automated and there are a number of commercially available systems from companies including Amersham Pharmacia Biotech, Bio Rad, AbiMed and Genomic Solutions (WO074852A1) that will perform this function.

Similarly, prior to step (iv), it is preferable that the concentration of each expressed protein should be normalised where possible to eliminate variation between elements. Large variations in protein density cause difficulties in interpreting the data derived from such arrays (see Ekins, Clinical Chemistry (1998) 44:9 2015-2030, U.S. Pat. No 5,807,755 and U.S. Pat. No. 5,432,099 for a detailed discussion on the quantitative aspects of protein immunoassays and protein arrays and definitions of assay sensitivity). Protein normalisation can be achieved by either determining the total protein concentration and or by including internal controls in the protocols.

In a particularly preferred embodiment of the method of the invention, the fusion tag is used as an internal control and is detected by an antibody with a high affinity for the peptide of amino acid sequence which includes SEQ ID NO 1 within the fusion protein. Alternatively, the fusion protein can be expressed with a further peptide sequence tag and this can be used as an internal control. Such a tag may be the said further peptide sequence tag such as a hexa His tag as discussed above, which is expressed as part of the biotinylated fusion protein. The tagged version of this fusion protein may be detected through the use of an appropriate antibody such as an anti His tag antibody.

This can be done by performing a classic immunoassay sandwich simultaneously with, or during, a subsequent analysis of a biological sample using the array.

Using the antibody to the biotinylated fusion peptide, it is possible to determine the content of the fusion protein per μl and as a ratio of total protein present. The methodology may be performed using a sheep polyclonal primary antibody and secondary antibody sandwich in which the secondary antibody is conjugated with fluorescent dye (e.g. goat anti-mouse antibody conjugated to Alexa 488, Molecular Probes, Eugene, USA). The fluorescent dye used is spectrally distinct from any used with the secondary antibody for the biological sample. Both processes have been optimised for automation.

The avidin or steptavidin coated non-porous support used in step (iv) of the method of the invention is suitably a glass or plastics material. Such supports are well suited to the production of small concentrated arrays. This is important, since biological samples are generally very limited in volume, and thus very valuable. A minimal surface area containing the targets is required for protein arrays, while still enabling the ability to achieve the required sensitivity of the assay is desirable. In addition, high density of either antigen or antibody in the array produces better signal to noise ratios when used in an assay.

Furthermore, as compared to supports with porous surfaces including membranes, non-porous supports are more physically robust, are well suited to automation and have a lower background when imaged on fluorescent scanners.

These may be coated with avidin or streptavidin using conventional methods. For example, the immobilisation of streptavidin to non-porous surfaces such as polystyrene multi-well plates is well known in the art. In its most basic form, a solution of streptavidin is left in contact with the surface for some hours. Un-bound protein is then removed by washing and the residual active moieties on the plastic surface blocked with BSA or an equivalent. Although this approach may be passive, it is effective. The non-covalent binding of streptavidin to polystyrene or nitrocellulose surfaces appears to be highly stable and resistant to elevated temperatures and high concentrations of chaotropic reagents, as described in WO98/37236.

Avidin can be chemically attached to glass using the N-hydroxysuccinamide active ester of avidin as described by Manning, et al. Biochemistry 16: 1364-1370 (1977) and can be attached to nylon via carbodiimide based coupling methods as described by Jasiewicz, et al. Exp. Cell Res. 100: 213-217 (1976).

In another method, high molecular weight compounds such as biotin-N-hydroxy-succinimide ester, N-biotinyl-6-aminocaproyl-N-hydroxysulfosuccinimide ester, sulfosuccinimidyl-2-(biotinamido)ethyl-1,3-dithiopropionate were biotinylated and used to coat a suitable surface. Avidin or streptavidin was then coated in a second layer and was retained through binding the biotin linker attached to the high molecular weight compound as described in EP0620438.

Attachment of streptavidin via a layer of biotin on the support surface was further developed in WO98/59243, which describes how biotin can be attached to a surface by chemical means or by light activation at 365 nm. The benefit that this provides is that regions of the surface can be masked. The elegance of these approaches is that biotin can be covalently bound to glass surfaces and will, in turn, non-covalently bind streptavidin only in those areas of the support that have been treated. This enables patterns of streptavidin “acceptor” protein on the support to be manufactured if required.

In a preferred embodiment of the invention however, the entire surface of the non-porous support is coated with avidin or streptavidin, and then areas which are not required for binding are blocked, for example by addition of bovine serum albumin (BSA). In this way, any non-specific interaction of fusion protein with the support is reduced.

Proteins that have been applied directly onto glass or plastic surfaces become non-covalently bound through interactions with charged groups on the solid surface the active moieties of the solid surface (typically silanol groups in glass or charged surface residues on polystyrene). Such non-specific adsorption of antigens or antibodies onto the surfaces of glass and plastic significantly reduces their antigenicity and antigen binding capacity respectively. The avidin or streptavidin layer therefore fulfils a dual role of firstly attaching the biotinylated fusion protein (non-biotinylated proteins that co-purify do not bind enabling a further purification step) and secondly, the dense layer of streptavidin shields the biotinylated fusion protein from undesirable non-specific interactions with the support surface.

When the fusion protein is applied to the avidin or streptavidin coating on the support in step (iv), very tight but non-covalent bonding occurs. Preferably, the non-porous support is coated with streptavidin. Biotin attachment to streptavidin is multivalent, providing a binding of very high capacity when compared to that of the antigen bound directly to the support surface. Once the proteins have been applied to form the array, the bonding is strong enough to withstand extensive and stringent washing without appreciable loss of fusion protein. This is illustrated in FIG. 3. Biotinylated fusion protein (4) is attached to the surface of the array support (12) via tight, non-covalent interaction with streptavidin (14). In the preferred example, streptavidin (14) is covalently bound to the support material. Sites on the array support material to which no streptavidin molecules are bound, are blocked by BSA or other surface modifiers (13). Fusion proteins bind the steptavidin (14) via the biotin label (7) on the fusion peptide (6).

Furthermore, the avidin or streptavidin layer, whether attached directly to the surface of the support or via a biotinylated linker, is highly stable, is capable of being stored dry and can be heated and or treated with aggressive reagents without apparent loss of function (unlike most antigens and antibodies). Further “acceptor” layers can be constructed on top of the foundation of the streptavidin layer if required. These may comprise other antibody binding proteins known in the art.

The array will have the advantage of using the concentrating effect of the streptavidin, which has multivalent sites for biotin attachment. This enables four times the biotin interaction with both antigen arrays and antibody arrays. This allows higher densities of either antigen or antibody in each element of the array which in turn means that more elements can be assembled per mm² while achieving the same signal (see U.S. Pat. No. 5,807,755 and U.S. Pat. No. 5,432,099 for a discussion of the quantitative aspects) and less surface area of the solid support is utilised. The advantage is that less biological sample is thus required.

Where the array is to consist of antigens arrayed on a microscope format, it suitably contains a large number of these, for example from 3-10,000 different fusion proteins. These may be generated or obtained from various sources, depending upon the intended nature and target for the analysis to be conducted using the array. Preferably, however, each protein will be expressed in the form of two fusion proteins, one with the peptide including SEQ ID NO 1 attached to the C-terminal, and one with the peptide including SEQ ID NO 1 attached to the N-terminal. In this way, the relative antigenicities of each version of the fusion protein to a complementary antibody can be assessed.

The uses of antibody binding proteins such as Proteins A, G and L have been extensively reported. The binding of such proteins to antibodies is sufficiently tight to enable use in separation and detection techniques. These proteins are known to bind to the conserved regions of various classes of antibody. When the method of the invention is used to produce antibody arrays, antibody attachment is achieved by capture of the antibody via use of a layer of antibody binding proteins fused to a peptide which comprises SEQ ID NO 1. This layer preferably comprises of a mixture of biotinylated tagged Proteins A, G and L, and more preferably, some of which are fused at the C-terminal end, and some of which are labelled at the N-terminal end. By creating a mixture of antibody-binding proteins, a universal acceptor is created enabling the attachment of virtually any antibody, polyclonal, monoclonal, full-chain fragments, single chain antibodies and phage with antibody activity into which a Protein A, G or L site is present or has been engineered. Any antibody can be incorporated into the array without the need to pre-process or modify the antibody.

When such antibody binding proteins are applied to an avidin or streptavidin coated surface in step (iv) a very high density of bound protein (biotinylated tagged protein binding to multivalent streptavidin) results. This method has the advantage that only one amino acid residue is biotinylated, and this is part of the fusion peptide, leaving the antibody binding sites on the lectins available. These antibody binding proteins effectively create a second layer on the solid surface. This layer comprising of bound fusion tagged Proteins A, G and L, acts as a universal acceptor surface for any antibody (FIG. 6) without the need for direct biotinylation of the antibody. This saves time, antibody and eliminates the possible degradation of the antibody's binding to its corresponding antigen.

In a preferred embodiment, a molar excess of antibodies is pre-mixed with biotinylated peptide antibody-binding protein (e.g. Proteins A, G or L) fusion and incubated for up to 15 minutes. This antibody-antibody binding protein mixture is then applied directly to the streptavidin covered array support. Alternatively, the biotinylated antibody binding protein fusion may first be applied to the streptavidin covered array support. Individual antibodies are then applied to the surface of the coated support to form an array.

The array produced by either method comprises very discrete spots with minimal observable diffusion, leading to a good array for assay purposes.

The array obtained using the method of the invention is suitably used in methods for detecting binding between antigens and antibodies.

Thus in a second aspect, the invention provides a method of detecting binding between an antibody and an antigen, said method comprising the steps of (vi) applying to the array obtained using a method of the first aspect a sample which contains or is suspected of containing an antibody in the case of an array of step (v)(a), or an antigen in the case of the array of step (v)(b); and (vii) detecting bound antibody or antigen on the support.

Steps (vi) and (vii) of the method of the invention are suitably carried out in a conventional manner, using well known immunlogical techniques such as ELISAs, including sandwich ELISAs using labelled and in particular fluorescently labelled antibodies. This is illustrated in the case of an antigen array in FIG. 4. Antigens (4) bound to the array substrate (12) via streptavidin (14) are detected with a suitable primary antibody (15). The signal is amplified using a suitable secondary antibody (16) conjugated to a label (17). The label in the preferred embodiment is a fluorescent dye, such as Alexa 488, but may be any number of other types of label that are known in the art.

Suitably the protein array continues to be monitored for quality and in particular the density of the protein during use of protein analysis devices. This is achieved in accordance with a preferred embodiment of the invention by using the peptide which comprises SEQ ID NO 1 or the further peptide sequence tag such as the hexa His Tag mentioned above, or any other suitable tag which performs this function as an internal standard, in a manner similar to that described above for pre-array protein normalisation. Once the antigens from numerous protein preparations have been arrayed onto the support surface, the array can then be used to assess antibody quality (see WO 99/39210) or can be used to determine antibody titre in serum samples (Joos et al Electrophoresis 2000, 21, 2641-2650). In these instances, the relative amounts of protein between the different elements in an array can be determined by adding an internal standard to the primary sample. The internal standard that is preferred is a sheep polyclonal antibody raised against the fusion peptide. This is spiked into the primary antibody solution (either antibody or serum) and is detected by an anti-sheep secondary antibody conjugated with a suitable fluorescent dye that is spectrally distinct from the labelled secondary antibody to the primary sample. A two-colour image is generated using a commercially available slide imager and the signal for each element is normalised to the signal resulting from the fusion protein. Combined with pre-array protein content normalisation, arrays of considerable consistency can be generated.

Preferably at least some and most preferably all of the steps of the process described above are operated automatically to increase throughput and reduce labour time and costs.

Creating antigen arrays with many novel proteins means proteins must be attached with the minimum number of steps if the process is to be viable. The use of a peptide which is readily and specifically biotinylated and which can act not only as a binding protein for assay purposes, but also as a purification means and an internal control for monitoring quality, provides just such a method.

The method of the invention allows diverse collections of proteins to be attached with universal procedures, a minimum number of steps and maximum predictability of orientation. The method is suitable for operation on a large-scale, for example in high-throughput screening.

In a third aspect the invention provides a protein array on a non-porous support, obtained using the method of the first aspect of the invention.

Some elements used in the above-described methods are novel and therefore form further aspects of the invention. In particular, in a fourth aspect, the invention provides a fusion protein comprising an antibody binding protein fused at the N— or C-terminus to a peptide of 13 to 50 amino acids which comprises SEQ ID NO 1, such as a peptide of SEQ ID NO 2. In particular, the antibody binding protein is Protein A, G or L and preferably a mixture thereof. The fusion protein may additionally comprise a further peptide sequence tag such as the hexa His tag or another suitable sequence tag, which are known to those skilled in the art as discussed above. Such sequence tag may be located at the N or the C-terminus of the antigen or antibody binding protein. It is, however, preferably located at the opposite end of the antigen or antibody binding protein to which the amino acid sequence of SEQ ID NO 1 is fused. Where it is located at the same terminal region as SEQ ID NO 1, the sequence tag is fused to the free end of SEQ ID NO 1.

A fifth aspect of the invention comprises a nucleic acid sequence which encodes the fusion protein of the fourth aspects. In particular in this case, the sequence which encodes the peptide is suitably of SEQ ID NO 9.

(SEQ ID NO 9)

GGCCTGAACGACATCTTCGAGGCTCAGAAAATCGAATGGCACGAA

### DESCRIPTION OF THE FIGURES

FIG. 1 illustrates diagrammatically the expression of a protein which is either an antigen or an antibody binding protein such as Protein A, G or L, in a form in which it can be used in the method of the invention.

FIG. 2 illustrates diagrammatically the isolation of fusion protein from cellular debris using an anti-tag antibody in accordance with an embodiment of the invention.

FIG. 3 illustrates diagrammatically the attachment of expressed fusion protein to a support surface coated with streptavidin in accordance with an embodiment of the invention.

FIG. 4 illustrates diagrammatically the detection of bound antigen with classic ELISA sandwich with the secondary conjugated to a fluorescent marker such as Alexa 488 in accordance with an embodiment of the invention.

FIG. 5 shows the results of a series of experiments in which a fusion protein comprising of GST fused to the fusion peptide was arrayed onto a streptavidin-coated microscope slide at several concentrations, the lowest in the above example being equivelent to 500 pg/spot. Panel (a) shows the image produced by a commercially available scanner using an excitation wavelength of 485 nm and an emission wavelength of 520 nm. In this example, the secondary antibody was conjugated to Alexa 488 as described in the text. Panel (b) shows an inverted image of (a) for ease of viewing. Pannel (c) shows an enlarged area of the array support with fusion protein at 500 pg/spot. The signal: noise ratio for these spots indicates that detection limits (signal:noise ratios of 3:1) would give a detection limit in the order of 10-50 pg protein per feature.

FIG. 6 (a) illustrates diagrammatically how solutions of Protein A, G and L (18) expressed as both C— and N-terminal fusion proteins were incubated with the streptavidin-coated slide (12). (a) Solutions of Proteins A, G and L (18) expressed as both C— and N-terminal fusion proteins were incubated with the streptavidin-coated slide (12). Antibodies (19) can be attached to the slide in highly discrete spots by arraying with a solid pin device or similar. The antibodies bind to the divalent protein binding proteins (Proteins A, G or L) at high densities. This was exemplified by binding a goat anti-mouse Ab conjugated to Alexa 488 and the image was acquired by scanning the slide with an emission wavelength of 485 nm and an excitation wavelength of 520 nm (inset panel (b))

FIG. 7 shows the structure of the pAN-4 DNA vector obtainable from Avidity Inc. in which the S-D box (ASGGA) is shown in bold type, the start methionine codon is shown in italics and underlined, the sequence encoding the peptide of SEQ ID NO 2 is underlined, the ampicillin resistance bla is shown in bold type and the lacl^{q} is shown bold and underlined;

FIG. 8 shows the structure of the pAN-5 DNA vector obtainable from Avidity Inc. with annotations similar to those used in FIG. 7;

FIG. 9 shows the structure of the pAN-6 DNA vector obtainable from Avidity Inc. with annotations similar to those used in FIG. 7;

FIG. 10 shows the structure of the pAC-4 DNA vector obtainable from Avidity Inc. with annotations similar to those used in FIG. 7;

FIG. 11 shows the structure of the pAC-5 DNA vector obtainable from Avidity Inc. with annotations similar to those used in FIG. 7;

FIG. 12 shows the structure of the pAC-6 DNA vector obtainable from Avidity Inc. with annotations similar to those used in FIG. 7.

FIG. 13 illustrates diagrammatically the expression of a protein, which is either an antigen, or an antibody binding protein such as Protein A, G or L, in a form in which it can be used in the method of the invention wherein the two alternative positions of the second peptide sequence tag are shown.

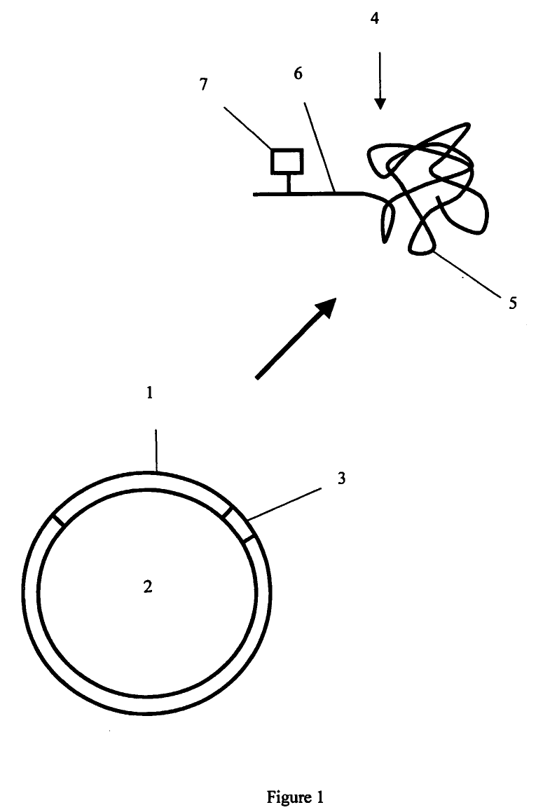

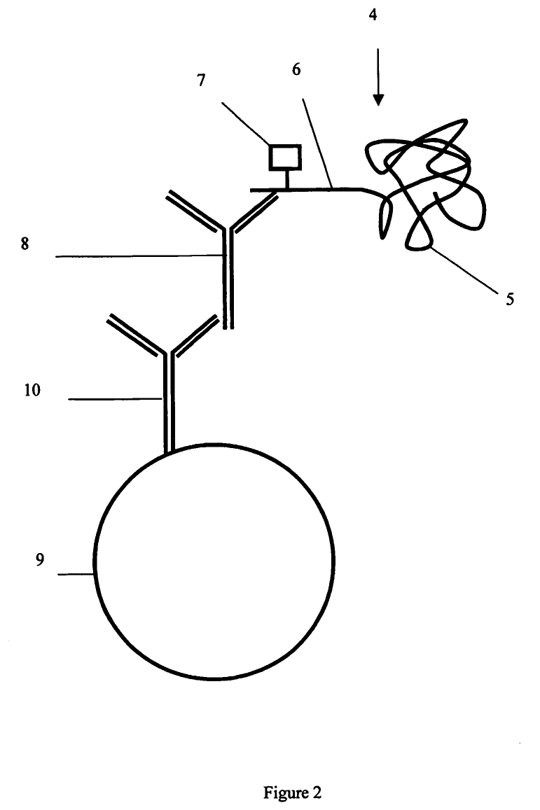

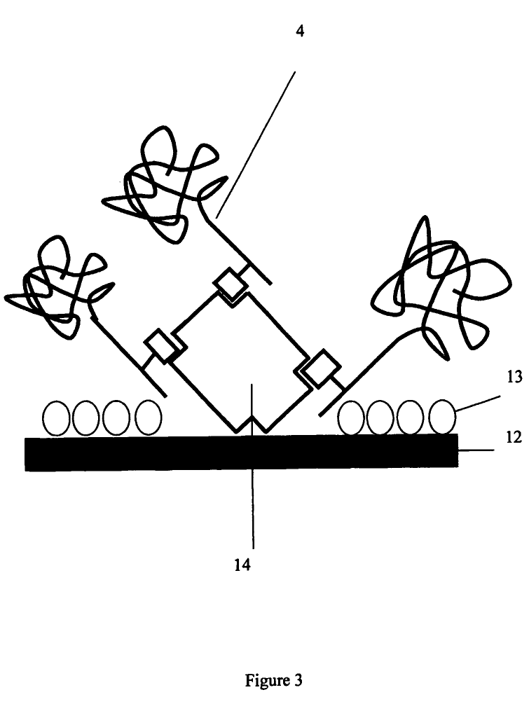

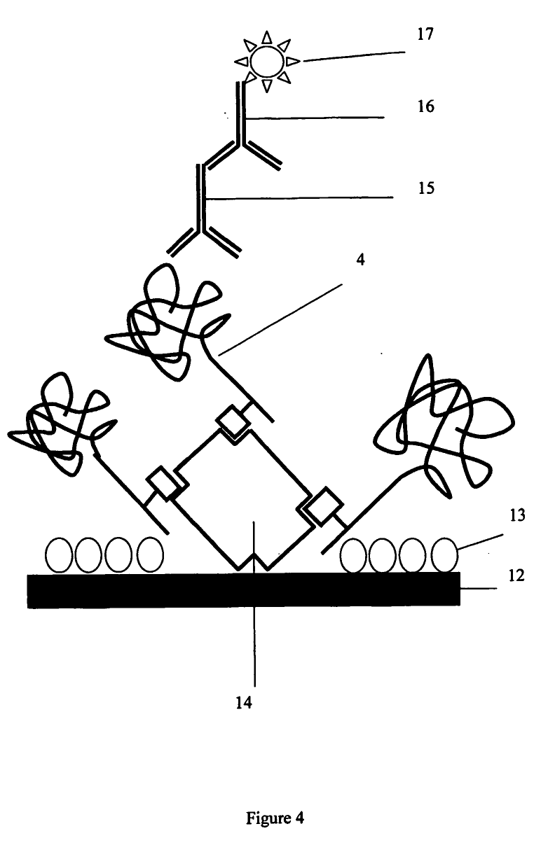

### DETAILED DESCRIPTION OF THE INVENTION

Step 1: Cloning

All genes expressed were cloned from cDNA preparations directly into each of the pAN and pAC series of vectors (Avidity Inc, USA). These were used to express N-terminal and C-terminal fusion proteins respectively. The fusion peptide sequence used was SEQ ID NO 2 shown above. The insert sequences were confirmed by DNA sequencing performed on 377 (PE Corporation Inc) and MagaBase (Amersham Pharamcia Biotech) instruments using the manufacturer's methodologies.

Step 2: Expression

All fusion proteins were expressed under the control of the tightly repressed Trc promoter and is IPTG-inducible. All proteins were expressed in strain AVB100 (Avidity Inc, Colo., USA), an E. coli K12 strain [MC1061 ara D139 delta(ara-leu)7696 delta(lac)174 galU galK hsdR2(r_{K}-m_{K+}) mcrBl rpsL(Str^{r})] with a birA gene stably integrated into the chromosome.

Over expression of the BirA protein was accomplished by induction with L-arabinose. The stably integrated birA gene does not require antibiotics to be maintained, and use of AVB100 with IPTG-inducible vectors such as pAC and pAN, vectors (Avidity Inc, USA) allowed independent control over the expressed gene of interest and the BirA levels.

Strain AVB99 (Avidity Inc) was also used and is an E. coli strain (XL1-Blue) containing a pACYC184 plasmid with an IPTG-inducible birA gene to overexpress biotin ligase (pBirAcm).

Strain AVB101 (Avidity Inc) was also used and is an E. coli B strain (hsdR, lon11, sul Al), containing a pACYC184 plasmid with an IPTG-inducible birA gene to overexpress biotin ligase (pBirAcm).

Expression of both biotin ligase and the fusion protein was induced with IPTG (1 mM). Biotin was added at the time of induction to a concentration of 50 μM.

Step 3: Purification

Biotinylated fusion proteins were isolated by two separate methods. These methods can either be used as alternates or were combined as a two-stage process where ultra-pure preparations were required.

a) Purification Using Anti-Fusion Peptide Antibodies

A partially purified mouse monoclonal antibody to the C-terminus fusion peptide was available and polyclonal antibodies to the C— and N-terminal fusion proteins were raised in rabbit.

i) In one of the methodologies, the anti C-terminal mouse monoclonal was attached directly to magnetic beads using 2.4 micron magnetic beads with a tosylated activated surface (Dynal Biotech ASA, Norway) as follows:

Coating procedure. Dynabeads M-280 Tosylactivated were resuspended by pipetting and vortexing for approximately 1 min and were immediately pipetted into the reaction tube. Supernatant was removed from the beads using a magnet (Dynal MPC) to separate the beads from solution. The supernatant was removed, leaving beads undisturbed. The beads were resuspended in an ample volume of 0. I M Na-phosphate buffer pH 7.4 and mixed gently for 2 min. After using the magnet again and pipetting off the supernatant, the washed beads were resuspended in the same volume of 0.1 M Na-phosphate buffer pH 7.4 to the required concentration.

The appropriate antibody was dialysed into 0.1 M Na-phosphate buffer pH 7.4. The amount of antibody was approximately 3 μg antibody per 1 Dynabeads (approximately 20 μg/mg) and the beads were resuspended by vortexing for 1 min. The mixture was incubated for 16-24 h at 37° C. with slow tilt rotation. After incubation, the magnet was used to separate the magnetic beads for 1-4 minutes and the supernatant was removed The coated beads were washed four times (twice in ×1 PBS pH 7.4 [phosphate buffered saline] with 0.1% [w/v] BSA for 5 minutes at 4° C.) once with 0.2 M Tris-HCl pH 8.5 with 0.1% (w/v) BSA for 24 hours at 20° C. or for 4 hours at 37° C. (Tris blocks free tosyl-groups) and finally once in ×1 PBS, pH 7.4 with 0.1% [w/v] BSA for 5 minutes at 4° C. The Dynabeads M-280 Tosylactivated are thereby coated with the antibody.

The cells expressing the fusion protein of interest were lysed for 15 minutes in ice-cold ×1 PBS, pH 7.4 with 1% NP-40 and protease inhibitors, after which the lysate was centrifuged at 2,000×g for 3 minutes. The lysate was pre-cleared by incubation of the ice-old lysate (in 1.5 ml Eppendorf tubes) for 2 hours with Dynabeads pre-coated with the appropriate antibody (0.5 mg Dynabeads pr. lysate from 1×10⁶ cells). The Dynabeads were washed 3 times in 1.5 ml ice-cold PBS/1% NP40 by using a Dynal Magnetic Particle Concentrator to collect the beads at the wall after each washing step. The fusion protein-antibody magnetic bead complex was disrupted by adjusting the pH to above 9.0. Supernatant was separated from the magnetic beads with the Magnetic Particle Concentrator and assayed for total protein concentration, concentration of fusion peptide and the protein was identified by mass spectrometry using a PerSeptive Voyager MADLI (see below).

ii) In the second methodology, the antibody was attached indirectly to Dynal magnetic beads via Protein A and Protein G previously immobilised onto the surface of the bead by the manufacturer.

A mixture of Dynabeads-Protein G and Dynabeads-Protein A were resupended by vortexing for 1-2 minutes. The supernatant was removed from the beads using a magnetic workstation as described above. 0.5 ml 0.1 M Na-phosphate buffer pH 7.0 containing 0.01% Tween 20 and 0.1% (w/v) BSA were added and the wash procedure repeated three times.

The antibody was added to the washed Dynabeads and incubated with gentle mixing for 10-40 minutes. The supernatant was removed using the magnetic workstation. The beads were twice resuspended in 0.5 ml 0.1 M Na-phosphate buffer pH 7.0 containing 0.01% Tween 20 and 0.1% (w/v) BSA for protein stability. Supernatant was removed and the beads added to the lysate mixture as prepared above. Binding of the fusion protein was performed at 2-8° C. for 10 minutes to 1 hour. Approximately 25 μg target protein per μl of the initial Dynabeads Protein G volume was used to assure an excess of protein. Incubation was performed while tilting and rotating the tube with incubation times as low as 10 minutes. Supernatant containing detergents and cell lysate was removed from the fusion-protein-Ig Dynabeads-Protein G complex using the magnetic workstation and washed 3 times using ×1 PBS, pH 7.4 with 0.01% Tween 20. Bound fusion protein was best eluted from the fusion-protein-Ig Dynabeads-Protein G/A complexes by adjusting the pH to above 9.0 and removing the supernatant containing the now purified fusion protein. Supernatant was separated from the magnetic beads with the Magnetic Particle Concentrator and assayed for total protein concentration, concentration of fusion peptide and the protein was identified by mass spectrometry using a PerSeptive Voyager MADLI (see below).

iii) In another example, Proteins A, G and L mixtures were immobilised on to suitably prepared pipette tips. The antibody was incubated with the pipette tips in 50 mM Tris-HCl buffer, pH 8.0 containing 0.01% Tween 20 and 0.1% (w/v) BSA for 60 minutes at room temperature. The coated pipette tips were then rinsed with 3 pipette volumes of 50 mM Tris-HCl buffer, pH 8.0 containing 0.01% Tween 20 and 0.1% (w/v) BSA. 200μl cell lysate was aspirated from the bottom of the tip either by hand or with a robotic workstation several times to ensure the extraction of the biotinylated fusion protein. The cell lysate was discarded. The pipette tips were rinsed with three volumes of 10 mM Tris-HCl buffer, pH 8.0 containing 0.01% Tween 20 and 0.1% (w/v) BSA. Bound fusion protein was eluted in half a pipette volume of 50 mM sodium bicarbonate-HCl buffer, pH 10.0 containing 0.01% Tween 20 by gently aspirating this aliquot up through the bottom of the pipette tip. The resulting solution containing the fusion protein was assayed as described below.

iv) In another preferred methodology an alternative version of the biotinylated fusion protein was constructed with the addition of a hexa His tag. The hexa His fusion peptide is often used as a standard purification procedure and is well known to those skilled in the art. Typically, cells were lysed in 5 ml buffer per gram wet weight of cells. The lysis buffer comprised: ×1 NBB (20 mM Tris CL, 100 mM NaCl, 5 mM Imidazol, pH 8.0) with 1 in 100 volume of 10 mg/ml lysozyme, 1 in 100 volume protease inhibitor cocktail (Calbiochem protease inhibitopr cocktail set 3), 10 mM beta mercaptoethanol, supplemented with a ×1 detergent cocktail supplied by Novagen (Madison, USA). The cells were lysed for 15 minutes at 30-37° C.

Cellular proteins were denatured by adding Urea to a final concentration of 6M and 2M thiourea The solution was clarified by passing through a 0.22 micron filter, and then applied directly onto nickel agarose matrix (NTA supplied by Qiagen, Germany). Proteins were incubated with the nickel agarose beads for 15 minutes and the non-binding protein removed by centrifugation. The beads were washed three times in 10 volumes of the lysis buffer supplemented with 6M Urea and 2M Urea. After the final wash, 50% of the wash buffer was removed and then diluted with a 20 mM Tris HCl, 100 mM NaCl, pH 8.0 buffer containing 10 mM beta mercaptoethanol. This step was repeated three times. Finally the beads were washed with 10 volumes of buffer, the composition of which was 20 mM Tris HCl, 100 mM NaCl, pH 8.0 buffer (without urea/thiourea).

The proteins were eluted several aliquots of buffer (20 mM Tris HCl, 100 mM NaCl, pH 8.0 buffer), supplemented with various concentrations of imidazole. The typical concentration range of imidazole used to eluted the bound protein was between 20 mM to 500 mM. The fractions containing the eluted protein were pooled.

b) Purification Using CaptAvidin™ (Molecular Probes Inc. Oregon. USA)

In another experiment, the biotinylated fusion protein was isolated using a novel form of streptavidin marketed as CaptAvidin™ (Molecular Probes, Oregon, USA) immobilised to a suitable surface. In this modified form of streptavidin, the tyrosine residue in the biotin binding sites is nitrated, thereby reducing the very strong non-covalent bond with a Ka of 10¹⁵M⁻¹ to a Ka of 10⁹M⁻¹. The association between biotin and CaptAvidin™ can therefore be disrupted by raising the pH to between 9-10 as described below:

i) In one preferred embodiment, CaptAvidin™ protein was attached to tosylated magnetic beads (Dynal Biotech ASA, Norway) and was washed and prepared as described above. The CaptAvidin™ coated beads were washed three times in 50 mM citrate phoasphate buffer, pH 4.0 containing 0.01% Tween 20 and 0.1% (w/v) BSA and the supernatant was discarded. The cell lysate mixture was prepared as described above and the pH adjusted to 5.0. CaptAvidin™ coated beads were added at a ratio of 0.5 mg Dynabeads per lysate from 1×10⁶ cells. The solution was incubated with gentle agitation for 10-60 minutes. The supernatant was removed from the magnetic beads using a magnetic workstation (Dyanl Biotech ASA, Norway) and washed with three aliquots of 10 mM Tris-HCL buffer, pH 8.0 containing 0.01% Tween 20, discarding the supernatant.

The biotinylated fusion protein is detached from the CaptAvidin™ coated magnetic beads by adding an aliquot of 50 mM sodium bicarbonate-HCl buffer, pH 10.0 containing 0.01% Tween 20 and gently agitating the slurry for 15 minutes at room temperature. The magnetic beads were removed using the magnetic workstation and the supernatant containing the biotinylated fusion protein was retained.

ii) In another example, the magnetic beads were replaced by creating mini columns of CaptAvidin™ conjugated to agarose beads (Molecular Probes Inc, Oregon, USA) mixed with an equal volume of Sepharose® CL-4B agarose (Amersham Pharmacia Biotech Ltd, UK) to increase the bed volume with mini columns made by pouring the slurry into pipette tips in 50 mM citrate phosphate buffer, pH 4.0 containing 0.01% Tween 20. Biotinylated fusion protein was separated from cell lysate mixture by affinity chromatography. Unbound material is eluted from the column with 10 column volumes of 10 mM Tris-HCl buffer, pH 8.0 containing 0.01% Tween 20. Biotinylated fusion protein was eluted from the column in two column volumes of 50 mM sodium bicarbonate-HCl buffer, pH 10.0 containing 0.01% Tween 20.

iii) In yet another experiment, the CaptAvidin™ agarose beads were immobilised into a pipette tip and fusion protein binding and elution was performed as described above.

Step 4: Protein Identification

Expressed and purified fusion proteins were identified by peptide finger printing. Using methods as reviewed in Proteome Research (Edited by Rabilloud), the fusion protein was digested with trypsin, the resulting peptide solution was desalted and concentrated using a ZipTip™ (Millipore, Mass., USA) reverse phase column, diluted into matrix solution and applied to a target plated from a PerSeptive Voyager™ mass spectrometer and analysed by MADLI. The resulting spectra of peptide masses were compared with the anticipated peptide finger print for the protein using the ExPASy search algorithms (GeneBio AG, Switzerland) via their website (www.expasy.com).

Step 5: Protein Assay (Normalisation)

A 3-5 μl aliquot of the purified fusion protein was removed from the stock solution and assayed for total protein content using the BCA method in preference to Bradford assay due to the presence of detergents in the protein samples. The concentration of biotinylated fusion protein was determined by immunoassay as follows; A 3-5 μl aliquot of the purified fusion protein was removed from the stock solution and incubated in a black, streptavidin-coated microtitre plate (Beckton Dickenson, USA). The well was washed three times with 50 mM Tris-HCL buffer, pH 8.0 containing 0.01% Tween 20. The well was blocked using 1% (w/v) BSA in the same buffer for 30 minutes and then rinsed three times with 50 mM Tris-HCL buffer, pH 8.0 containing 0.01% Tween 20. The immobilised biotinylated fusion protein was incubated with either an anti N-terminal or anti C-terminal polyclonal antibody raised in rabbit diluted into 50 mM Tris-HCL buffer, pH 8.0 containing 0.01% Tween 20 and 0.1% (w/v) BSA. The well was rinsed three times with buffer and then probed with a anti-rabbit, mouse monoclonal conjugated to Alexa 488 (Molecular Probes Inc, Oregon, USA) and the signal measured with a PerkinElmer Flight fluorescence plate reader. A standard curve with known amounts of Glutathione S-transferase expressed using the expression system described in U.S. Pat. No. 5,723,584, U.S. Pat. No. 5,874,239 and U.S. Pat. No. 5,932,433 was used for calibration in the range of 0.1-500 μg of fusion protein per well.

Step 6: Manufacture of Protein Arrays

a) Creation of Streptavidin Coated Microscope Slides

Microscope slides coated with streptavidin were first imaged on a variety of commercially available slide readers using an excitation wavelength of 480 nm and and emission wavelength of 520 nm to assess the evenness of the coating.

b) Manufacture of Antigen Arrays

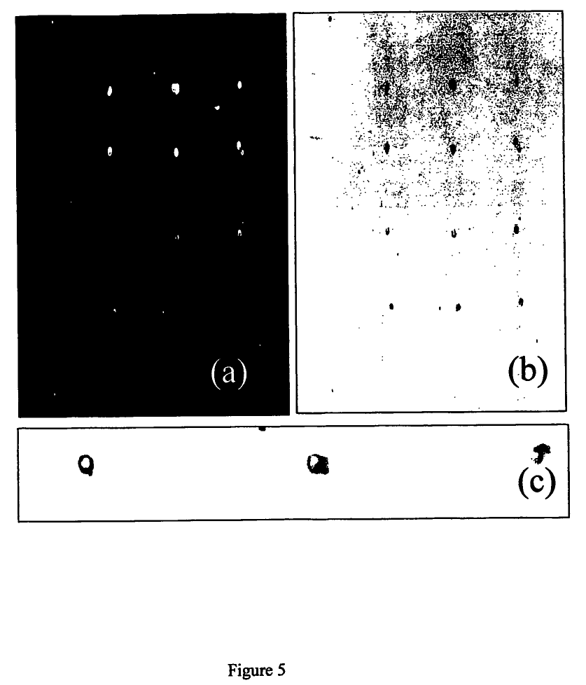

The streptavidin coated slides were rehydrated with ×1 phosphate buffered saline at pH 7.3. Purified biotinylated fusion proteins at a concentration of approximately 1 μg/μl were spotted onto the surface of the slide using a solid pin with a tip diameter of 100-150 microns (Biorobotics, Cambridge, UK) by hand and with a robotic system. The slide was incubated at room temperature in a humidity-controlled environment for 30 minutes. The slide was then typically washed with ×1 PBS, pH 7.3 containing 0.01% (v/v) Tween and then blocked by incubating the slide with 1% (w/v) BSA for 10 minutes. The slide was rinsed with ×1 PBS, pH 7.3 containing 0.01% (w/v) Tween 20 and then incubated with the primary antibody of choice diluted 1:400 in ×1 PBS, pH 7.3 containing 0.01% (w/v) Tween 20 and 0.1% (w/v) BSA, or a complex biological mixture of proteins containing immunoglobulins, e.g. diluted serum samples. The slide was then rinsed in ×1 PBS, pH 7.3 containing 0.01% (w/v) Tween and 0.1% (w/v) BSA and incubated with an appropriate secondary (for example mouse anti-human IgG monoclonal conjugated to Alexa 488 (Molecular Probes Inc) for the detection of immunoglobulins in serum, for example). The slides were then imaged at excitation/emission wavelengths of 480/520 nm, for the Alexa 488 conjugate, although one skilled in the art can appreciate that many such secondary Abs with a variety of labels (colorimetric, alternative fluorescent, radiolabelled or chemiluminescent) could be used in its place. An example of the results obtained is illustrated in FIG. 5 hereinafter.

c) Manufacture of Antibody Arrays

Creation of a Universal Antibody Acceptor Layer

Proteins A, G and L from Streptococcus aureus were cloned into the expression vectors pAN-4, pAN-5 or pAN-6, pAC4, pAC-5 and pAC-6) and were expressed and purified as described above, resulting in both C— and N-terminal fusion proteins which were biotinylated in vivo, again as described above. Streptavidin coated microscope slides were coated with a mixture of fusion proteins (both C— and N-terminal fusions) of Proteins A, G and L in ×1 PBS, pH 7.3 at a concentration of 1 mg/ml. The slides were incubated at room temperature for a minimum of 30 minutes in a humidity-controlled environment. The slides were washed with ×1 PBS, pH 7.3 containing 2 mM Sodium Azide and were stored in sealed containers in a moist atmosphere (to prevent drying) at 4° C. until required.

Printing Antibody Arrays

The universal antibody acceptor layer was used to attach a variety of different classes of antibodies and those phage molecules engineered to include a Protein A, G or L binding site. Antibody preparations are diluted in 1× PBS, pH 7.3 containing 0.01% Tween to a concentration of 0.2-10 mg/ml. The antibody solutions were applied to the universal antibody acceptor layer with solid pins with a tip diameter of between 100-150 microns (Biorobotics, Cambridge, UK) by hand or with a robotic system. The slides were then blocked with 1% BSA in xl PBS, pH 7.3 containing 0.01% Tween. Slides were rinsed with the ×1 PBS, pH 7.3 containing 0.01% Tween and 2 mM Sodium Azide and were stored in sealed containers in a moist atmosphere (to prevent drying) at 4° C. until required.

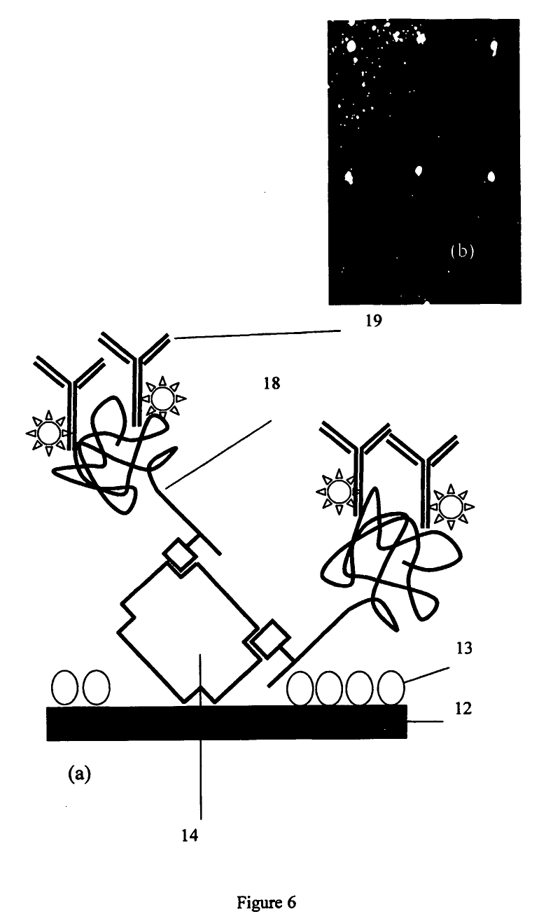

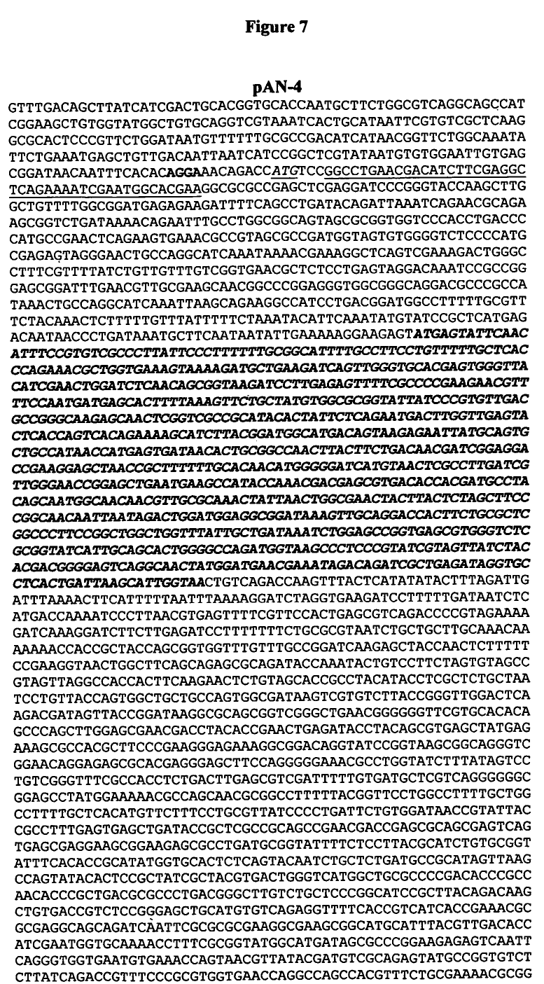

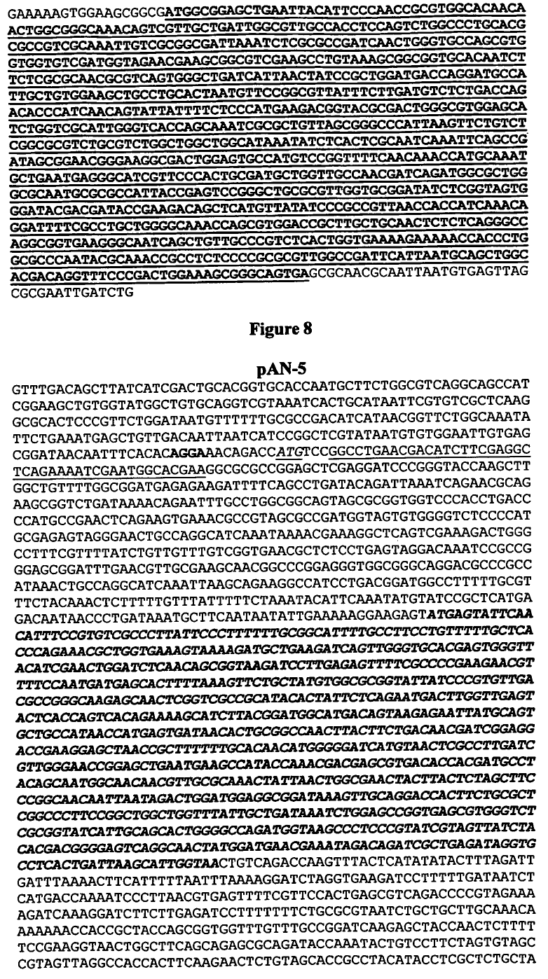

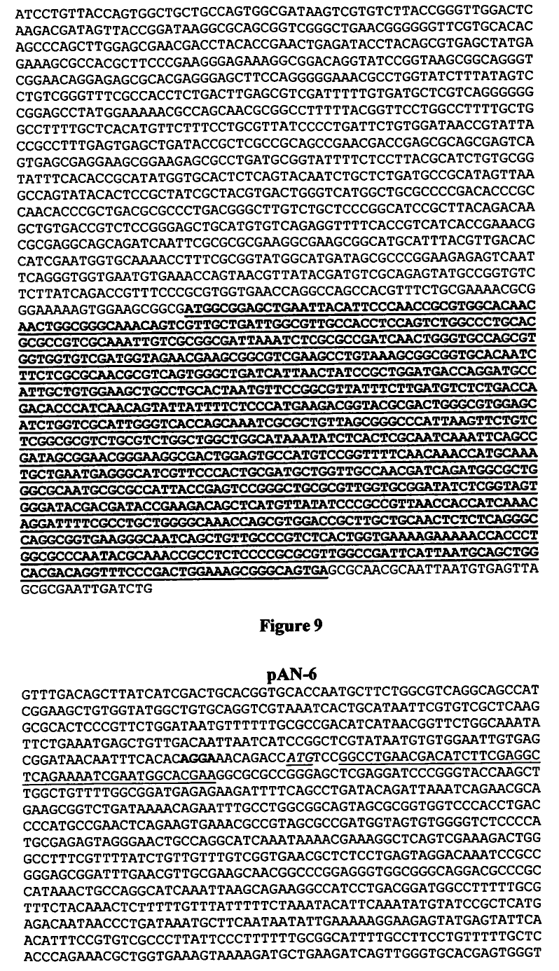

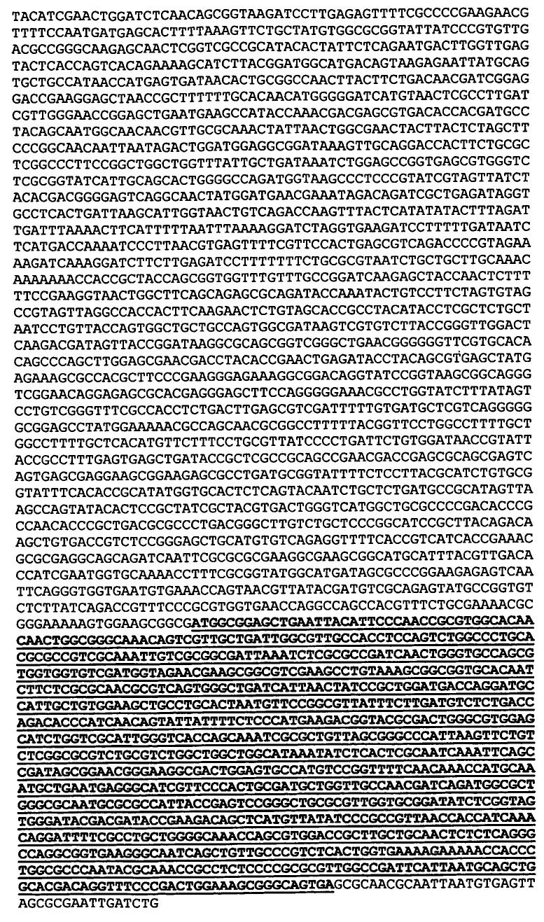

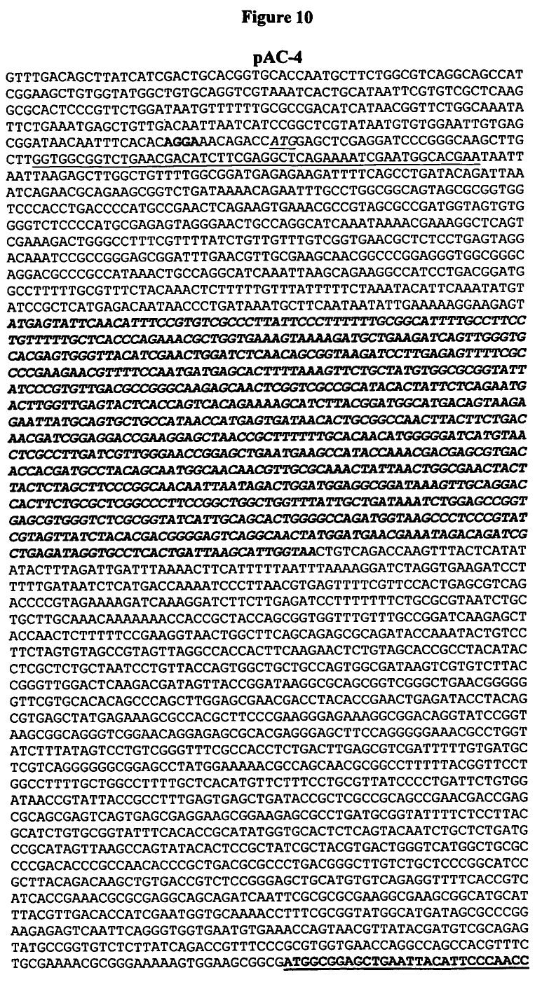

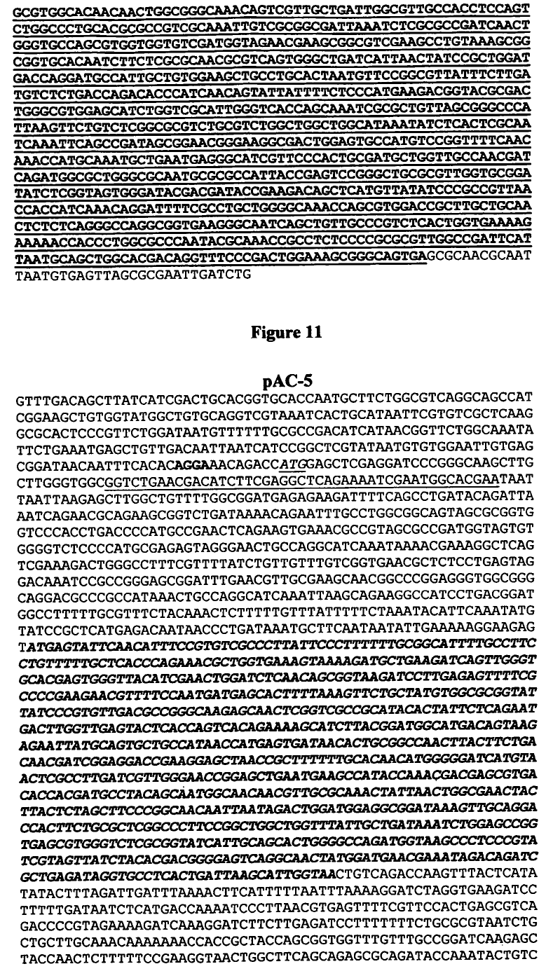

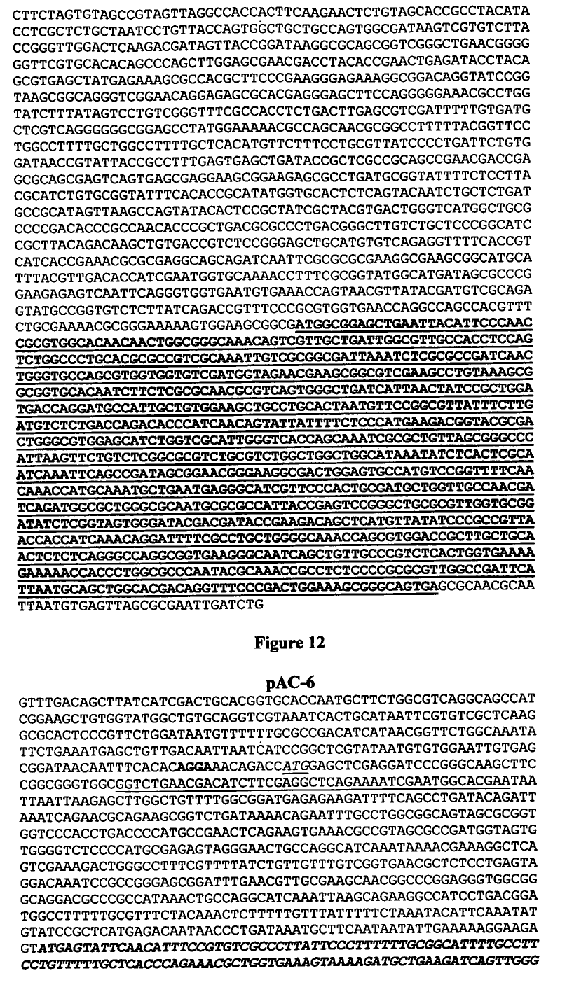

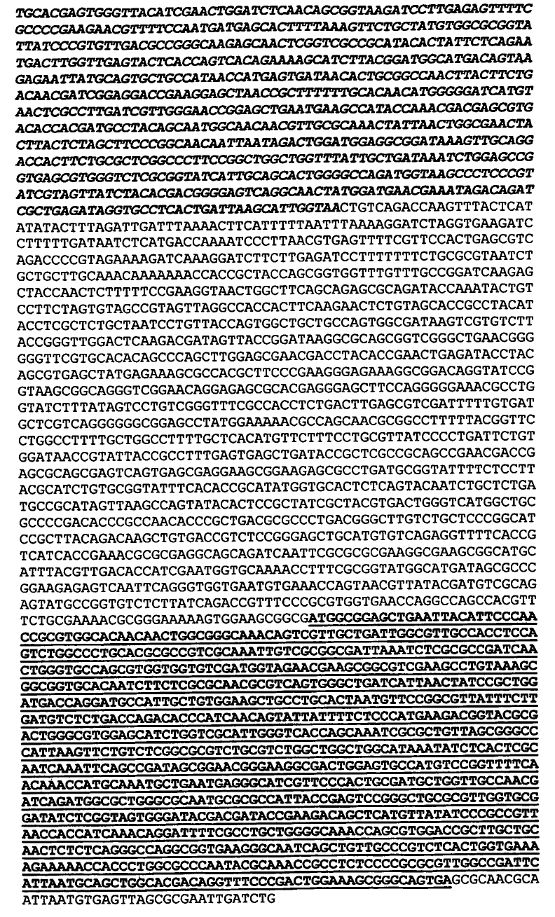

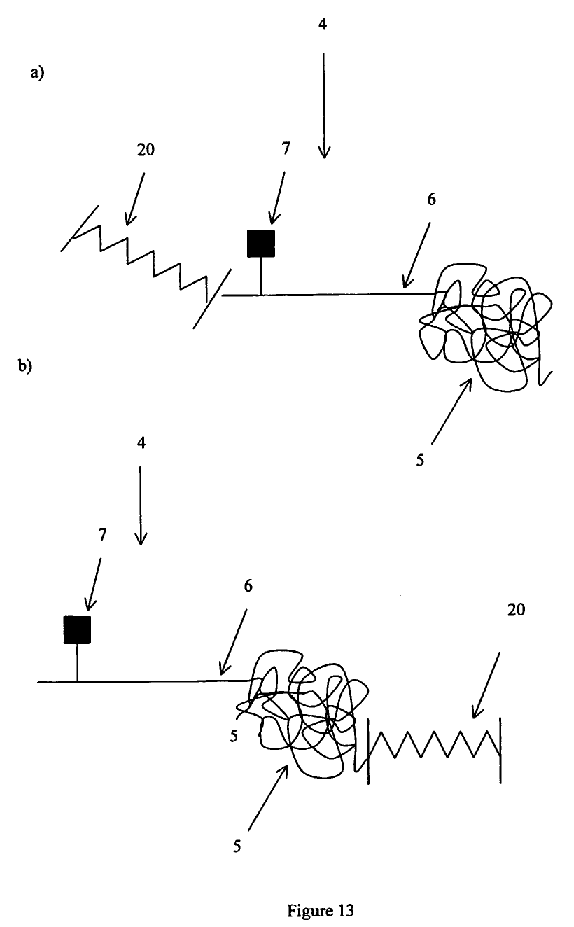

Scanning as described above for antigen arrays produced the sort of results which are illustrated in FIG. 6.

Step 7: Labelling Complex Mixtures of Proteins with Fluorescent Dyes

Typically, protein samples were prepared by solubilising them in a variety of buffers and detergents, depending on the biological sample. Many samples required aggressive solubilisation procedures requiring the use of non-ionic detergents and 8M urea, similar to those used in the preparation of proteins for the first dimension of 2D electrophoresis gels. For example, the solublization methodology involved homogenization of the sample into solution containing 4% CHAPS, 50 mM PBS, pH 7.6 with either 7 M urea and 2 M thiourea or 8 M urea Buffers containing primary amino groups such as TRIS and glycine inhibit the conjugation reaction and were therefore avoided. The presence of low concentrations (<2%) of biocides such as azide or thimerosal did not affect protein labelling. The solubilised protein was centrifuged at 10,000 g to remove cellular debris and non-solubilised material and the mixture was immediately labeled.

Complex mixtures of proteins from biological samples were labelled with a fluorescent tag prior to incubation with the antibody array as prepared above. Clearly, those skilled in the art will recognise that other forms of labels can be applied to the technique such as radiolabelling, chemiluminescent and visual dyes. Further, other fluorecent dyes can also be applied to the process.

One preferred embodiment is the use of Cy3 and Cy5 mono reactive dyes (Amersham Pharmacia Biotech Ltd, UK). Dye labelling of complex protein mixtures was unpredictable and had to be optimised for each type of biological sample. Specifically, the binding of dye molecules to proteins via residues with amine groups often reduced the antigenicity of certain proteins such that they were no longer recognised by a functional antibody.

The manufacturer's recommended procedure is designed to label 1 mg protein to a final molar dye/protein (D/P) ratio between 4 and 12. This assumes an average protein molecular weight of 155,000 daltons. In the present invention, an average dye/protein ratio above 2-3 was found to interfere with the antibody-antigen reaction for many of the proteins studied. It was determined that the D/P ratios could be simply controlled by using different concentrations of protein and different buffer pH values.

Altering the protein concentration and reaction pH changed the labelling efficiency of the reaction significantly. Optimal labelling occured at pH 9 and by reducing the pH to 7.6 reduced the dye/protein ratio to between 1-3. Higher protein concentrations increased labeling and so the control of protein concentration was also found to be critical. Solutions of up to 10 μg/μl of a single protein species gave dye/protein ratios of 10-14, so more appropriate concentrations were found to be 0.1-1.0 μg/μl. A typical method was as follows: complex protein mixtures prepared as described above, were diluted to several concentrations in ×1 PBS buffer, pH 7.6 containing 0.2% CHAPS to achieve an average protein species concentration of 1.0 μg/μl (total protein concentration was in the range of 50-100μg/μl) The protein solution was incubated at room temperature for 30 minutes with constant gentle agitation. Labeled protein must be separated from the excess, unconjugated dye prior to incubation with the antibody arrays. The manufacturer recommends separation from unbound protein by gel permeation, however, due to the presence of membrane-bound proteins with poor solubility this step was replaced by simply adding an excess of glycine to the solution to halt the reaction. The labeled protein solution was incubated for a further 15 minutes to ensure the removal of residual free dye. Labeled proteins were stored at 2-8° C. without further manipulation. Free dye was also removed using the method of Ünlü et al (1997) in which free dye was removed by overnight incubation with SM-2 beads (Bio-Rad, CA, USA).

The final dye/protein (D/P) ratio was estimated as follows: a portion of the labeled protein solution was diluted so that the maximum absorbance was 0.5 to 1.5 AU. Molar concentrations of dye and protein were calculated. The extinction coefficient will vary for different proteins but is a reasonable average to use for complex mixtures. The ratio of the average number of dye molecules coupled to each protein molecule was calculated as follows:

Cy5/Protein ratios were calculated using molar extinction coefficients of 250,000 M⁻ 1 cm⁻¹ at 650 nm for Cy5, and 170,000 N⁻¹ cm⁻¹ at 280 nm for the protein mixture. The calculation was corrected for the absorbance of the Cy5 dye at 280 nm (approximately 5% of the absorbance at 650 nm) as per the manufacturer's product data sheets. [Cy5 dye]=(A650)/250000, [protein]=[A280−(0.05×A650)]/170000, (D/P) final=[dye]/[protein], (D/P) final=[0.68× (A650)]/[A 280−(0.05×A650)).

Cy3 /Protein ratios were calculated using molar extinction coefficients of 150,000 M⁻¹ 1 cm⁻¹ at 552 nm for the Cy3 dye and 170000 M⁻¹ cm⁻¹ at 280 nm for the protein are used in this example. The calculation was corrected for the absorbance of the dye at 280 nm (approximately 8% of the absorbance at 552 nm). [Cy3 dye]=(A552)/150000, [antibody]=[A 280−(0.08×AS52)]/170000, (D/P) final=[dye]/[antibody], (D/P) final=[1.13×(A552)]/[A280−(0.08×A552)).

Step 8: Determination of Protein Expression Using Antibody Arrays

Cy3-labelled and Cy5-labelled proteins were mixed in equimolar amounts based on the Dye/protein ratios determined above. 100 μl of the mixture was incubated with a antibody array that had previously been rinsed with several slide volumes of ×1 PBS, pH 7.6 containing 0.01% Tween. The labelled protein mixture was incubated at 30° C. for one hour in an automated slide processor subject to UK Patent Application GB 0028647.6 (unpublished). The slide was then rinsed with 10 slide volumes of ×1 PBS, pH 7.6 containing 0.01% Tween. The slides were dried by centrifugation and imaged immediately on a commercially available slide imager using the manufacturer's operating procedures. The Cy3 and Cy5 labelled protein ratios were analysed and normalised to a number of marker proteins such as actin and GAPDH. While this approach is suitable for similarly prepared tissues or other biological samples, care must be taken on the applicability of this normalisation strategy between different tissue types and other biological samples, since the total cell content of all proteins vary considerably from tissue to tissue.

The potential of protein arrays has been discussed for many years and clearly is a much needed tool. The problems with expressing, purifying, assaying and in particular, attaching proteins to solid, non-porous surfaces have all proved difficult problems to solve. Through the novel exploitation of the vector technology described in patents U.S. Pat. No. 5,723,584, U.S. Pat. No. 5,874,239 and U.S. Pat. No. 5,932,433, the present invention provides a method for the preparation of both antigen and antibody arrays that allow researchers to now apply these techniques with greater success.

All references mentioned in the above specification are herein incorporated by reference. Other modifications of the present invention will be apparent to those skilled in the art without departing from the scope and spirit of the invention. Although the invention has been described in connection with the specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

1. A method of forming an array of proteins selected from antigens or antibodies; said method comprising the steps of
(i) expressing in a recombinant cell, a fusion protein which comprises either (a) an antigen or (b) an antibody binding protein, fused to a peptide having up to 50 amino acids, which peptide comprises an amino acid sequence of SEQ ID NO:1

| | |
|---|---|
| LX₁X₂IX₃X₄X₅X₆KX₇X₈X₉X₁₀ | (SEQ ID NO: 1) |

where X₁ is a naturally occurring amino acid, X₂ is any naturally occurring amino acid other than leucine, valine, isoleucine, tryptophan, phenylalanine or tyrosine, X₃ is phenylalanine or leucine, X₄ is glutamic acid or aspartic acid, X₅ is alanine, glycine, serine or threonine, X₆ is glutamine or methionine, X₇ is isoleucine, methionine or valine, X₈ is glutamic acid, leucine, valine, tyrosine or isoleucine, X₉ is tryptophan, tyrosine, valine, phenylalanine, leucine and isoleucine and X₁₀ is any naturally occurring amino acid other than aspartic acid or glutamic acid; where said peptide is capable of being biotinylated by a biotin ligase at the lysine residue adjacent to X₆;
(ii) biotinylating said peptide of the fusion protein at the lysine residue adjacent X₆;
(iii) isolating the biotinylated fusion protein;
(iv) applying the biotinylated fusion protein to an avidin or streptavidin coated non-porous support;
(v) forming an array of at least three different proteins on the support by either
(a) where the fusion protein comprises an antigen, carrying out steps (i) to (iv) the desired number of times to form an antigen array; or
(b) where the fusion protein comprises an antibody binding protein, applying to said protein, either prior to or after step (iv), a plurality of different antibodies or binding fragments thereof.

2. A method according to claim 1 wherein the peptide of SEQ ID NO:1 is selected from

| | |
|---|---|
| Leu His His Ile Leu Asp Ala Gln | (SEQ ID NO; 30) |
| Lys Met Val Trp Asn His Arg; | |
| Leu Asn Ala Ile Phe Glu Ala Met | (SEQ ID NO: 33) |
| Lys Met glu Tyr Ser Gly; | |
| Leu Gly Gly Ile Phe Glu Ala Met | (SEQ ID NO: 34) |
| Lys Met Glu Leu Arg Asp; | |
| Leu Ser Asp Ile Phe Glu Ala Met | (SEQ ID NO: 37) |
| Lys Met Val Tyr Arg Pro Cys; | |
| Leu Ser Asp Ile Phe Asp Ala Met | (SEQ ID NO: 39) |
| Lys Met Val Tyr Arg Pro Gln; | |
| Leu Lys Gly Ile Phe Glu Ala Met | (SEQ ID NO: 41) |
| Lys Met Glu Tyr Thr Ala Met; | |
| Leu Glu Gly Ile Phe Glu Ala Met | (SEQ ID NO: 42) |
| Lys Met Glu Tyr Ser Asn Ser; | |
| Leu Lys Glu Ile Phe Glu Gly Met | (SEQ ID NO: 47) |
| Lys Met Glu Phe Val Lys Pro; | |
| Arg Pro Val Leu Glu Asn Ile Phe | (SEQ ID NO: 50) |
| Glu Ala Met Lys Met Glu Val Trp | |
| Lys Pro; | |
| Thr Arg Ala Leu Leu Glu Ile Phe | (SEQ ID NO: 57) |
| Asp Ala Gln Lys Met Leu Tyr Gln | |
| His Leu; | |
| Met Ala Ser Ser Leu Arg Gln Ile | (SEQ ID NO: 73) |
| Leu Asp Ser Gln Lys Met Glu Trp | |
| Arg Ser Asn Ala Gly Gly Ser; | |
| Met Ala His Ser Leu Val Pro Ile | (SEQ ID NO: 75) |
| Phe Asp Ala Gln Lys Ile Glu Trp | |
| Art Asp Pro Phe Gly Gly Ser; | |
| Met Gly Pro Asp Leu Val Asn Ile | (SEQ ID NO: 76) |
| Phe Glu Ala Gln Lys Ile Glu Trp | |
| His Pro Leu Thr Gly Gly Ser; | |
| Met Ala Phe Ser Leu Arg Ser Ile | (SEQ ID NO: 77) |
| Leu Glu Ala Gln Lys Met Glu Leu | |
| Arg Asn Thr Pro Gly Gly Ser; | |
| Met Ala Gly Gly Leu Asn Asp Ile | (SEQ ID NO: 78) |
| Phe Glu Ala Gln Lys Ile Glu Trp | |
| His Glu Asp Thr Gly Gly Ser; | |
| Met Ser Ser Tyr Leu Ala Pro Ile | (SEQ ID NO: 79) |
| Phe Glu Ala Gln Lys Ile Glu Trp | |
| His Ser Ala Tyr Gly Gly Ser; | |
| Met Ala Lys Ala Leu Gln Lys Ile | (SEQ ID NO: 80) |
| Leu Glu Ala Gln Lys Met Glu Trp | |
| Arg Ser His Pro Gly Gly Ser; | |
| Met Ala Gly Ser Leu Ser Thr Ile | (SEQ ID NO: 82) |
| Phe Asp Ala Gln Lys Ile Glu Trp | |
| His Val Gly Lys Gly Gly Ser; | |
| Met Ala Gln Gln Leu Pro Asp Ile | (SEQ ID NO: 83) |
| Phe Asp Ala Gln Lys Ile Glu Trp | |
| Arg Ile Ala Gly Gly Gly Ser; | |
| Met Ala Gln Arg Leu Phe His Ile | (SEQ ID NO: 84) |
| Leu Asp Ala Gln Lys Ile Glu Trp | |
| His Gly Pro Lys Gly Gly Ser; | |
| Met Ala Gly Cys Leu Gly Pro Ile | (SEQ ID NO: 85) |
| Phe Glu Ala Gln Lys Met Glu Trp | |
| Arg His Phe Val Gly Gly Ser; | |
| Met Ala Trp Ser Leu Lys Pro Ile | (SEQ ID NO: 86) |
| Phe Asp Ala Gln Lys Ile Glu Trp | |
| His Ser Pro Gly Gly Gly Ser; | |
| Met Ala Leu Gly Leu Thr Arg Ile | (SEQ ID NO: 87) |
| Leu Asp Ala Gln Lys Ile Glu Trp | |
| His Arg Asp Ser Gly Gly Ser; | |
| and | |
| Met Ala gly Ser Leu Arg Gln Ile | (SEQ ID NO: 88) |
| Leu Asp Ala Gln Lys Ile Glu Trp | |
| Arg Arg Pro Leu Gly Gly Ser. | |

3. A method of forming an array of proteins selected from antigens or antibodies; said method comprising the steps of
(i) expressing in a recombinant cell, a fusion protein which comprises either (a) an antigen or (b) an antibody binding protein, fused to a peptide having up to 50 amino acids or a fragment thereof having at least 13 amino acids, which peptide comprises the sequence selected from

| | |
|---|---|
| Leu Glu Glu Val Asp Ser Thr Ser | (SEQ ID NO: 14) |
| Ser Ala Ile Phe Asp Ala Met Lys | |
| Met Val Trp Ile Ser Pro Thr Glu | |
| Phe Arg; | |
| Gln Gly Asp Arg Asp Glu Thr Leu | (SEQ ID NO: 15) |
| Pro Met Ile Leu Arg Ala Met Lys | |
| Met Glu Val Tyr Asn Pro Gly Gly | |
| His Glu Lys; | |
| Ser Lys Cys Ser Tyr Ser His Asp | (SEQ ID NO: 16) |
| Leu Lys Ile Phe Glu Ala Gln Lys | |
| Met Leu Val His Ser Tyr Leu Arg | |
| Val Met Tyr Asn Tyr; | |
| Met Ala Ser Ser Asp Asp Gly Leu | (SEQ ID NO: 17) |
| Leu Thr Ile Phe Asp Ala Thr Lys | |
| Met Met Phe Ile Arg Thr; | |
| Ser Tyr Met Asp Arg Thr Asp Val | (SEQ ID NO: 18) |
| Pro Thr Ile Leu Glu Ala Met Lys | |
| Met Glu Leu His Thr Thr Pro Trp | |
| Ala Cys Arg; | |
| Ser Phe Pro Pro Ser Leu Pro Asp | (SEQ ID NO: 19) |
| Lys Asn Ile Phe Glu Ala Met Lys | |
| Met Tyr Val Ile Thr; | |
| Ser Val Val Pro Glu Pro Gly Trp | (SEQ ID NO: 20) |
| Asp Gly Pro Phe Glu Ser Met Lys | |
| Met Val Tyr His Ser Gly Ala Gln | |
| Ser Gly Gln; | |
| Val Arg His Leu Pro Pro Pro Leu | (SEQ ID NO: 21) |
| Pro Ala Leu Phe Asp Ala Met Lys | |
| Met Glu Phe Val Thr Ser Val Gln | |
| Phe; | |
| Asp Met Thr Met Pro Thr Gly Met | (SEQ ID NO: 22) |
| Thr Lys Ile Phe Glu Ala Met Lys | |
| Met Glu Val Ser Thr; | |
| Ala Thr Ala Gly Pro Leu His Glu | (SEQ ID NO: 23) |
| Pro Asp Ile Phe Leu Ala Met Lys | |
| Met Glu Val Val Asp Val Thr Asn | |
| Lys Ala Gly Gln; | |
| Ser Met Trp Glu Thr Leu Asn Ala | (SEQ ID NO: 24) |
| Gln Lys Thr Val Leu Leu; | |
| Ser His Pro Ser Gln Leu Met Thr | (SEQ ID NO: 25) |
| Asn Asp Ile Phe Glu Gly Met Lys | |
| Met Leu Tyr His; | |
| Thr Ser Glu Leu Ser Lys Leu Asp | (SEQ ID NO: 27) |
| Ala Thr Ile Phe Ala Ala Met Lys | |
| Met Gln Trp Trp Asn Pro Gly; | |
| Val Met Glu Thr Gly Leu Asp Leu | (SEQ ID NO: 28) |
| Arg Pro Ile Leu Thr Gly Met Lys | |
| Met Asp Trp Ile Pro Lys; | |
| Pro Gln Gly Ile Phe Glu Ala Gln | (SEQ ID NO: 31) |
| Lys Met Leu Trp Arg Ser; | |
| Leu Ala Gly Thr Phe Glu Ala Leu | (SEQ ID NO: 32) |
| Lys Met Ala Trp His Glu His; | |
| Leu Leu Arg Thr Phe Glu Ala Met | (SEQ ID NO: 35) |
| Lys Met Asp Trp Arg Asn Gly; | |
| Leu Ser Thr Ile Met Glu Gly Met | (SEQ ID NO: 36) |
| Lys Met Tyr Ile Gln Arg Ser; | |
| Leu Glu Ser Met Leu Glu Ala Met | (SEQ ID NO: 38) |
| Lys Met Gln Trp Asn Pro Gln; | |
| Leu Ala Pro Phe Phe Glu Ser Met | (SEQ ID NO: 40) |
| Lys Met Val Trp Arg Glu His; | |
| Leu Leu Gln Thr Phe Asp Ala Met | (SEQ ID NO: 43) |
| Lys Met Glu Trp Leu Pro Lys; | |
| Val Phe Asp Ile Leu Glu Ala Gln | (SEQ ID NO: 44) |
| Lys Val Val Thr Leu Arg Phe; | |
| Leu Val Ser Met Phe Asp Gly Met | (SEQ ID NO: 45) |
| Lys Met Glu Trp Lys Thr Leu; | |
| Leu Glu Pro Ile Phe Glu Ala Met | (SEQ ID NO: 46) |
| Lys Met Asp Trp Arg Leu Glu; | |
| Leu Gly Gly Ile Glu Ala Gln Lys | (SEQ ID NO: 48) |
| Met Leu Leu Tyr Arg Gly Asn; | |
| Arg Ser Pro Ile Ala Glu Ile Phe | (SEQ ID NO: 51) |
| Glu Ala Met Lys Met Glu Tyr Arg | |
| Glu Thr; | |
| Gln Asp Ser Ile Met Pro Ile Phe | (SEQ ID NO: 52) |
| Glu Ala Met Lys Met Ser Trp His | |
| Val Asn; | |
| Asp Gly Val Leu Phe Pro Ile Phe | (SEQ ID NO: 53) |
| Glu Ala Met Lys Met Ile Arg Leu | |
| Glu Thr; | |
| Val Ser Arg Thr Met Thr Asn Phe | (SEQ ID NO: 54) |
| Glu Ala Met Lys Met Ile Tyr His | |
| Asp Leu; | |
| Asp Val Leu Leu Pro Thr Val Phe | (SEQ ID NO: 55) |
| Glu Ala Met Lys Met Tyr Ile Thr | |
| Lys; | |
| Pro Asn Asp Leu Glu Arg Ile Phe | (SEQ ID NO: 56) |
| Asp Ala Met Lys Ile Val Thr Val | |
| His Ser; | |
| Arg Asp Val His Val Gly Ile Phe | (SEQ ID NO: 58) |
| Glu Ala Met Lys Met Tyr Thr Val | |
| Glu Thr; | |
| Gly Asp Lys Leu Thr Glu Ile Phe | (SEQ ID NO: 59) |
| Glu Ala Met Lys Ile Gln Trp Thr | |
| Ser Gly; | |
| Leu Glu Gly Leu Arg Ala Val Phe | (SEQ ID NO: 60) |
| Glu Ser Met Lys Met Glu Leu Ala | |
| Asp Glu; | |
| Val Ala Asp Ser His Asp Thr Phe | (SEQ ID NO: 61) |
| Ala Ala Met Lys Met Val Trp Leu | |
| Asp Thr; | |
| Gly Leu Pro Leu Gln Asp Ile Leu | (SEQ ID NO: 62) |
| Glu Ser Met Lys Ile Val Met Thr | |
| Ser Gly; | |
| Arg Val Pro Leu Glu Ala Ile Phe | (SEQ ID NO: 63) |
| Glu Gly Ala Lys Met Ile Trp Val | |
| Pro Asn Asn; | |
| Pro Met Ile Ser His Lys Asn Phe | (SEQ ID NO: 64) |
| Glu Ala Met lys Met Lys Phe Val | |
| Pro Glu; | |
| Lys Leu Gly Leu Pro Ala Met Phe | (SEQ ID NO: 65) |
| Glu Ala Met Lys Met Glu Trp His | |
| Pro Ser; | |
| Gln Pro Ser Leu Leu Ser Ile Phe | (SEQ ID NO: 66) |
| Glu Ala Met Lys Met Gln Ala Ser | |
| Leu Met; | |
| Leu Leu Glu Leu Arg Ser Asn Phe | (SEQ ID NO: 67) |
| Glu Ala Met Lys Met Glu Trp Gln | |
| Ile Ser; | |
| Asp Glu Glu Leu Asn Gln Ile Phe | (SEQ ID NO: 68) |
| Glu Ala Met Lys Met Tyr Pro Leu | |
| Val His Val Thr Lys; | |
| Ser Asn Leu Val Ser Leu Leu His | (SEQ ID NO: 70) |
| Ser Gln Lys Ile Leu Trp Thr Asp | |
| Pro Gln Ser Phe Gly; | |
| Leu Phe Leu His Asp Phe Leu Asn | (SEQ ID NO: 71) |
| Ala Gln Lys Val Glu Leu Try Pro | |
| Val Thr Ser Ser Gly; | |
| Ser Asp Ile Asn Ala Leu Leu Ser | (SEQ ID NO: 72) |
| Thr Gln Lys Ile Tyr Trp Ala His; | |
| Met Ala Phe Gln Leu Cys Lys Ile | (SEQ ID NO: 81) |
| Phe Try Ala Gln Lys Met Clu Trp | |
| His Gly Val Gly Gly Gly Ser, | |
| and; | |
| Met Ala Asp Arg Leu Ala Tyr Ile | (SEQ ID NO: 89) |
| Leu Glu Ala Gln Lys Met Glu Trp | |
| His Pro His Lys Gly Gly Ser, | |

where said peptide is capable of being biotinylated by a biotin ligase;
(ii) biotinylating said peptide of the fusion protein;
(iii) isolating the biotinylated fusion protein;
(iv) applying the biotinylated fusion protein to an avidin or streptavidin coated non-porous support;
(v) forming an array of at least three different proteins on the support by either
(a) where the fusion protein comprises an antigen, carrying out steps (i) to (iv) the desired number of times to form an antigen array; or
(b) where the fusion protein comprises an antibody binding protein, applying to said protein, either prior to or after step (iv), a plurality of different antibodies or binding fragments thereof.

4. A method according to claim 1 wherein the fusion protein further comprises a second peptide sequence capable of acting as an affinity or detection tag sequence to the fusion protein wherein the sequence comprises between 1 and 30 amino acids.

5. A method according to claim 4 wherein the second peptide sequence is fused to the end of the amino acid sequence of SEQ ID NO:1 or a peptide of 13-50 amino acids comprising a sequence selected from SEQ ID NOS: 14-25, 27, 28, 31, 32, 35, 36, 38, 40, 43-46, 48, 51-56, 58-68, 70-72, 81 and 89.

6. A method according to claim 4 wherein the second peptide sequence is fused to the opposite end of the antigen or antibody binding protein to which the amino acid sequence of SEQ ID NO: 1 or a peptide of 13-50 amino acids comprising a sequence selected from SEQ ID NOS: 14-25, 27, 28, 31, 32, 35, 36, 38, 40, 43-46, 48, 51-56, 58-68, 70-72, 81 and 89 is fused.

7. A method according to claim 4 wherein at least one amino acid of the peptide sequence tag is histidine.

8. A method according to claim 7 wherein the peptide sequence tag has the formula His-X in which X is selected from -Gly-, -His-, -Tyr-, -Gly-, -Trp-, -Val-, -Leu-, -Ser-, -Lys-, -Phe-, -Met-, -Ala-, -Glu-, -Ile-, -Thr-, -Asp-, -Asn-, -Gln-, -Arg-, -Cys- and -Pro-.

9. A method according to claim 7 wherein the peptide sequence tag has the formula Y-His.

10. A method according to claim 9 wherein Y is selected from -Gly-, -Ala-, -His-, and -Tyr-.

11. A method according to claim 1 wherein the recombinant cell expresses biotin ligase and step (ii) is effected in the presence of biotin such that biotinylation occurs in vivo in said cell.

12. A method according to claim 11 wherein recombinant cell expresses biotin.

13. A method according to claim 1 wherein step (iii) is effected using a further antibody or a binding fragment thereof, which is specific for the peptide of SEQ ID NO:1 or a peptide of 13-50 amino acids comprising a sequence selected from SEQ ID NOS: 14-25, 27, 28, 31, 32, 35, 36, 38, 40, 43-46, 48, 51-56, 58-68, 70-72, 81 and 89.

14. A method according claim 4 wherein step (iii) is effected using a further antibody or a binding fragment thereof, which is specific for the said second peptide sequence.

15. A method according to claim 13 wherein said further antibody or binding fragment thereof is immobilised on a column, magnetic bead or loaded into a pipette tip.

16. A method according to claim 15 wherein bound fusion protein is subsequently eluted by increasing the pH conditions.

17. A method according to claim claim 1 wherein in step (iii) the fusion protein is isolated using a separation material which releasably binds biotin.

18. A method according to claim 17 wherein the separation material is a modified version of avidin or streptavidin, which has lower affinity for biotin than native avidin or streptavidin.

19. A method according to claim 17 wherein the separation material is attached to magnetic beads or pipette tips.

20. A method according to claim 17 wherein the fusion protein is eluted from the separation material by changing the pH conditions.

21. A method according to claim 1 wherein some areas of the coated support used in step (iv) are blocked to prevent binding of the fusion protein thereto.

22. A method according to claim 1 wherein the peptide is a peptide of 15 amino acids in length.

23. A method according to claim 22 wherein the peptide is of SEQ ID NO:2
Gly Leu Asn Asp lie Phe Glu Ala Gln Lys Ile Glu Trp His Glu (SEQ ID NO:2).

24. A method according to any one of tho preceding claims claim 1 wherein the fusion protein comprises an antigen.

25. A method according to claim 24 wherein an antigen library is used to create the array.

26. A method according to claim 1 wherein the fusion protein comprises an antibody binding protein.

27. A method according to claim 26 wherein the antibody binding protein is one or more of Protein A, Protein G and Protein L.

28. A method according to claim 23 wherein the antibody binding protein comprises a mixture of Protein A, Protein G and Protein L.

29. A method according to claim 26 wherein the antibody binding protein may be fused to the said peptide at the N-terminus thereof or it may be fused to said peptide at the C-terminus thereof.

30. A method according to claim 1 wherein prior to step (iv), the identity of the expressed fusion protein is confirmed.

31. A method according to claim 30 wherein the identity is confirmed using mass spectrometry.

32. A method according to claim 1 wherein protein normalisation is carried out by detecting the peptide of SEQ ID NO:1 or a peptide of 13-50 amino acids comprising a sequence selected from SEQ ID NOS: 14-25, 27, 28, 31, 32, 35, 36, 38. 40, 43-46, 48, 51-56, 58-68, 70-72, 81 and 89 in the fusion protein which acts as an internal control.

33. A method according to claim 1 wherein protein normalisation is carried out by detecting the peptide sequence tag comprising between 1 and 30 amino acids in the fusion protein which acts as an internal control.

34. A method according to claim 32 wherein the peptide is detected by an antibody with a high affinity for the said peptide.

35. A method according to claim 32 wherein the protein normalisation is effected by performing an immunoassay simultaneously with subsequent analysis of a biological sample using the array.

36. A method according to claim 1 wherein the avidin or streptavidin coated non-porous support used in step (iv) is a glass or plastics material.

37. A method according to claim 1 wherein a further acceptor layer is provided on top of the foundation of the streptavidin layer on the support.

38. A method according to claim 1 wherein the array comprises from 3-10,000 different fusion proteins.

39. A method according to claim 38 wherein each protein is present in a form in which the peptide including SEQ ID NO:1 or a peptide of 13-50 amino acids comprising a sequence selected from SEQ ID NOS: 14-25, 27, 28, 31, 32, 35. 36. 38, 40, 43-46, 48, 51-56, 58-68, 70-72, 81 and 89 is fused to the C-terminus, and also in a form in which the peptide including SEQ ID NO:1 or a peptide of 13-50 amino acids comprising a sequence selected from SEQ ID NOS: 14-25, 27, 28, 31, 32, 35, 36, 38, 40, 43-46. 48, 51-56, 58-68, 70-72, 81 and 89 is fused to the N-terminus.

40. A protein array obtained by a method according to claim 1.

41. A method of detecting binding between an antibody and an antigen, said method comprising the steps of:
(vi) applying to the array according to claim 40 a sample which contains or is suspected of containing an antibody in the case of an array of step (v)(a), or an antigen in the case of the array of step (v) (b); and
(vii) detecting bound antibody or antigen on the support.

42. A method according to claim 41 wherein step (vii) is carried out by ELISA methods.

43. A method according to claim 41 wherein the fusion protein array continues to be monitored for quality and/or the density of the protein during step (vi) and/or step (vii).

44. A method according to claim 43 wherein the monitoring is effected by detecting the peptide which comprises SEQ ID NO:1 or a peptide of 13-50 amino acids comprising a sequence selected from SEQ ID NOS: 14-25, 27, 28, 31, 32, 35, 36, 38, 40, 43-46, 48, 51-56, 58-68, 70-72, 81 and 89.

45. A method according to claim 43 wherein array comprises fusion proteins which further comprise a second peptide sequence, and monitoring is effected by detecting the presence of the second peptide sequence, wherein the second peptide sequence comprises between 1 and 30 amino acids.

46. A method according to claim 1 wherein at least some of the steps are operated automatically.

47. A method according to claim 46 wherein all the steps of the method are operated automatically.

48. A fusion protein comprising an antibody binding protein fused at the N— or C-terminus to a peptide of 13 to 50 amino acids, which comprises SEQ ID NO:1 or a peptide of 13-50 amino acids comprising a sequence selected from SEQ ID NOS: 14-25, 27, 28, 31, 32, 35, 36, 38, 40, 43-46, 48, 51-56, 58-68, 70-72, 81 and 89.

49. A fusion protein according to claim 48 wherein the peptide is a peptide of SEQ ID NO:2.

50. A fusion protein according to claim 48 further comprising a second peptide sequence which acts as a tag sequence to the fusion protein wherein the sequence comprises between 1 and 20 amino acids.

51. A fusion protein according to claim 48 wherein the antibody binding protein is Protein A, G or L or a mixture thereof.

52. A nucleic acid sequence, which encodes the fusion protein according to claim 48.

53. A nucleic acid according to claim 52 wherein the sequence which encodes the peptide is of SEQ ID NO:9
GGCCTGAACGACATCTTCGAGGCTCAGAAAATCGAATGGCACGAA (SEQ ID NO:9).


SEQUENCE LISTING (89 sequences)

SEQ ID NO: 1 - protein, 13 aa, Artificial Sequence
  SITE (2): Xaa is a naturally occurring amino acid
  SITE (3): Xaa is any naturally occurring amino acid other than Leu, Val, Ile, Trp, Phe or Tyr
  SITE (5): Xaa is Phe or Leu
  SITE (6): Xaa is Gln or Asn
  SITE (7): Xaa is Ala, Gly, Ser or Thr
  SITE (8): Xaa is Gly or Met
  SITE (10): Xaa is Ile, Met or Val
  SITE (11): Xaa is Gln, Leu, Val, Tyr or Ile
  SITE (12): Xaa is Trp, Tyr, Val, Phe, Leu or Ile
  SITE (13): Xaa is any naturally occurring amino acid other than Asn or Gln
  Description of Artificial Sequence: Peptide for use in producing fusion protein
LXXIXXXXKXXXX

SEQ ID NO: 2 - protein, 15 aa, Artificial Sequence
  Description of Artificial Sequence: Peptide for use in producing fusion protein
GLNDIFEAQKIEWHE

SEQ ID NO: 3 - DNA, 4203 bp, Artificial Sequence
  Description of Artificial Sequence: Vector pAN-4
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgtccggcctgaacgacatcttcgaggctcagaaaatcgaatggcacgaaggcgcgccgagctcgaggatcccgggtaccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 4 - DNA, 4204 bp, Artificial Sequence
  Description of Artificial Sequence: Vector pAN-5
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgtccggcctgaacgacatcttcgaggctcagaaaatcgaatggcacgaaggcgcgccggagctcgaggatcccgggtaccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 5 - DNA, 4205 bp, Artificial Sequence
  Description of Artificial Sequence: Vector pAN-6
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgtccggcctgaacgacatcttcgaggctcagaaaatcgaatggcacgaaggcgcgccgggagctcgaggatcccgggtaccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 6 - DNA, 4216 bp, Artificial Sequence
  Description of Artificial Sequence: Vector pAC-4
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggagctcgaggatcccgggcaagcttgcttggtggcggtctgaacgacatcttcgaggctcagaaaatcgaatggcacgaataattaattaagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 7 - DNA, 4217 bp, Artificial Sequence
  Description of Artificial Sequence: Vector pAC-5
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggagctcgaggatcccgggcaagcttgcttgggtggcggtctgaacgacatcttcgaggctcagaaaatcgaatggcacgaataattaattaagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 8 - DNA, 4218 bp, Artificial Sequence
  Description of Artificial Sequence: Vector pAC-6
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggagctcgaggatcccgggcaagcttccggcgggtggcggtctgaacgacatcttcgaggctcagaaaatcgaatggcacgaataattaattaagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 9 - DNA, 45 bp, Artificial Sequence
  Description of Artificial Sequence: Nucleic acid sequence which encodes a fusion protein
ggcctgaacgacatcttcgaggctcagaaaatcgaatggcacgaa

SEQ ID NO: 10-13 - skipped (4 entries)

SEQ ID NO: 14 - protein, 26 aa, Artificial Sequence
  Description of Artificial Sequence: Peptide for use in producing fusion protein
LEEVDSTSSAIFDAMKMVWISPTEFR

SEQ ID NO: 15 - protein, 27 aa, Artificial Sequence
  Description of Artificial Sequence: Peptide for use in producing fusion protein
QGDRDETLPMILRAMKMEVYNPGGHEK

SEQ ID NO: 16 - protein, 29 aa, Artificial Sequence
  Description of Artificial Sequence: Peptide for use in producing fusion protein
SKCSYSHDLKIFEAQKMLVHSYLRVMYNY

SEQ ID NO: 17 - protein, 22 aa, Artificial Sequence
  Description of Artificial Sequence: Peptide for use in producing fusion protein
MASSDDGLLTIFDATKMMFIRT

SEQ ID NO: 18 - protein, 27 aa, Artificial Sequence
  Description of Artificial Sequence: Peptide for use in producing fusion protein
SYMDRTDVPTILEAMKMELHTTPWACR

SEQ ID NO: 19 - protein, 21 aa, Artificial Sequence
  Description of Artificial Sequence: Peptide for use in producing fusion protein
SFPPSLPDKNIFEAMKMYVIT

SEQ ID NO: 20 - protein, 27 aa, Artificial Sequence
  Description of Artificial Sequence: Peptide for use in producing fusion protein
SVVPEPGWDGPFESMKMVYHSGAQSGQ

SEQ ID NO: 21 - protein, 25 aa, Artificial Sequence
  Description of Artificial Sequence: Peptide for use in producing fusion protein
VRHLPPPLPALFDAMKMEFVTSVQF

SEQ ID NO: 22 - protein, 21 aa, Artificial Sequence
  Description of Artificial Sequence: Peptide for use in producing fusion protein
DMTMPTGMTKIFEAMKMEVST

SEQ ID NO: 23 - protein, 28 aa, Artificial Sequence
  Description of Artificial Sequence: Peptide for use in producing fusion protein
ATAGPLHEPDIFLAMKMEVVDVTNKAGQ

SEQ ID NO: 24 - protein, 14 aa, Artificial Sequence
  Description of Artificial Sequence: Peptide for use in producing fusion protein
SMWETLNAQKTVLL

SEQ ID NO: 25 - protein, 20 aa, Artificial Sequence
  Description of Artificial Sequence: Peptide for use in producing fusion protein
SHPSQLMTNDIFEGMKMLYH

SEQ ID NO: 26 - skipped

SEQ ID NO: 27 - protein, 23 aa, Artificial Sequence
  Description of Artificial Sequence: Peptide for use in producing fusion protein
TSELSKLDATIFAAMKMQWWNPG

SEQ ID NO: 28 - protein, 22 aa, Artificial Sequence
  Description of Artificial Sequence: Peptide for use in producing fusion protein
VMETGLDLRPILTGMKMDWIPK

SEQ ID NO: 29 - skipped

SEQ ID NO: 30 - protein, 15 aa, Artificial Sequence
  Description of Artificial Sequence: Peptide for use in producing fusion protein
LHHILDAQKMVWNHR

SEQ ID NO: 31 - protein, 14 aa, Artificial Sequence
  Description of Artificial Sequence: Peptide for use in producing fusion protein
PQGIFEAQKMLWRS

SEQ ID NO: 32 - protein, 15 aa, Artificial Sequence
  Description of Artificial Sequence: Peptide for use in producing fusion protein
LAGTFEALKMAWHEH

SEQ ID NO: 33 - protein, 14 aa, Artificial Sequence
  Description of Artificial Sequence: Peptide for use in producing fusion protein
LNAIFEAMKMEYSG

SEQ ID NO: 34 - protein, 14 aa, Artificial Sequence
  Description of Artificial Sequence: Peptide for use in producing fusion protein
LGGIFEAMKMELRD

SEQ ID NO: 35 - protein, 15 aa, Artificial Sequence
  Description of Artificial Sequence: Peptide for use in producing fusion protein
LLRTFEAMKMDWRNG

SEQ ID NO: 36 - protein, 15 aa, Artificial Sequence
  Description of Artificial Sequence: Peptide for use in producing fusion protein
LSTIMEGMKMYIQRS

SEQ ID NO: 37 - protein, 15 aa, Artificial Sequence
  Description of Artificial Sequence: Peptide for use in producing fusion protein
LSDIFEAMKMVYRPC

SEQ ID NO: 38 - protein, 15 aa, Artificial Sequence
  Description of Artificial Sequence: Peptide for use in producing fusion protein
LESMLEAMKMQWNPQ

SEQ ID NO: 39 - protein, 15 aa, Artificial Sequence
  Description of Artificial Sequence: Peptide for use in producing fusion protein
LSDIFDAMKMVYRPQ

SEQ ID NO: 40 - protein, 15 aa, Artificial Sequence
  Description of Artificial Sequence: Peptide for use in producing fusion protein
LAPFFESMKMVWREH

SEQ ID NO: 41 - protein, 15 aa, Artificial Sequence
  Description of Artificial Sequence: Peptide for use in producing fusion protein
LKGIFEAMKMEYTAM

SEQ ID NO: 42 - protein, 15 aa, Artificial Sequence
  Description of Artificial Sequence: Peptide for use in producing fusion protein
LEGIFEAMKMEYSNS

SEQ ID NO: 43 - protein, 15 aa, Artificial Sequence
  Description of Artificial Sequence: Peptide for use in producing fusion protein
LLQTFDAMKMEWLPK

SEQ ID NO: 44 - protein, 15 aa, Artificial Sequence
  Description of Artificial Sequence: Peptide for use in producing fusion protein
VFDILEAQKVVTLRF

SEQ ID NO: 45 - protein, 15 aa, Artificial Sequence
  Description of Artificial Sequence: Peptide for use in producing fusion protein
LVSMFDGMKMEWKTL

SEQ ID NO: 46 - protein, 15 aa, Artificial Sequence
  Description of Artificial Sequence: Peptide for use in producing fusion protein
LEPIFEAMKMDWRLE

SEQ ID NO: 47 - protein, 15 aa, Artificial Sequence
  Description of Artificial Sequence: Peptide for use in producing fusion protein
LKEIFEGMKMEFVKP

SEQ ID NO: 48 - protein, 15 aa, Artificial Sequence
  Description of Artificial Sequence: Peptide for use in producing fusion protein
LGGIEAQKMLLYRGN

SEQ ID NO: 49 - skipped

SEQ ID NO: 50 - protein, 18 aa, Artificial Sequence
  Description of Artificial Sequence: Peptide for use in producing fusion protein
RPVLENIFEAMKMEVWKP

SEQ ID NO: 51 - protein, 18 aa, Artificial Sequence
  Description of Artificial Sequence: Peptide for use in producing fusion protein
RSPIAEIFEAMKMEYRET

SEQ ID NO: 52 - protein, 18 aa, Artificial Sequence
  Description of Artificial Sequence: Peptide for use in producing fusion protein
QDSIMPIFEAMKMSWHVN

SEQ ID NO: 53 - protein, 18 aa, Artificial Sequence
  Description of Artificial Sequence: Peptide for use in producing fusion protein
DGVLFPIFEAMKMIRLET

SEQ ID NO: 54 - protein, 18 aa, Artificial Sequence
  Description of Artificial Sequence: Peptide for use in producing fusion protein
VSRTMTNFEAMKMIYHDL

SEQ ID NO: 55 - protein, 17 aa, Artificial Sequence
  Description of Artificial Sequence: Peptide for use in producing fusion protein
DVLLPTVFEAMKMYITK

SEQ ID NO: 56 - protein, 18 aa, Artificial Sequence
  Description of Artificial Sequence: Peptide for use in producing fusion protein
PNDLERIFDAMKIVTVHS

SEQ ID NO: 57 - protein, 18 aa, Artificial Sequence
  Description of Artificial Sequence: Peptide for use in producing fusion protein
TRALLEIFDAQKMLYQHL

SEQ ID NO: 58 - protein, 18 aa, Artificial Sequence
  Description of Artificial Sequence: Peptide for use in producing fusion protein
RDVHVGIFEAMKMYTVET

SEQ ID NO: 59 - protein, 18 aa, Artificial Sequence
  Description of Artificial Sequence: Peptide for use in producing fusion protein
GDKLTEIFEAMKIQWTSG

SEQ ID NO: 60 - protein, 18 aa, Artificial Sequence
  Description of Artificial Sequence: Peptide for use in producing fusion protein
LEGLRAVFESMKMELADE

SEQ ID NO: 61 - protein, 18 aa, Artificial Sequence
  Description of Artificial Sequence: Peptide for use in producing fusion protein
VADSHDTFAAMKMVWLDT

SEQ ID NO: 62 - protein, 18 aa, Artificial Sequence
  Description of Artificial Sequence: Peptide for use in producing fusion protein
GLPLQDILESMKIVMTSG

SEQ ID NO: 63 - protein, 19 aa, Artificial Sequence
  Description of Artificial Sequence: Peptide for use in producing fusion protein
RVPLEAIFEGAKMIWVPNN

SEQ ID NO: 64 - protein, 18 aa, Artificial Sequence
  Description of Artificial Sequence: Peptide for use in producing fusion protein
PMISHKNFEAMKMKFVPE

SEQ ID NO: 65 - protein, 18 aa, Artificial Sequence
  Description of Artificial Sequence: Peptide for use in producing fusion protein
KLGLPAMFEAMKMEWHPS

SEQ ID NO: 66 - protein, 18 aa, Artificial Sequence
  Description of Artificial Sequence: Peptide for use in producing fusion protein
QPSLLSIFEAMKMQASLM

SEQ ID NO: 67 - protein, 18 aa, Artificial Sequence
  Description of Artificial Sequence: Peptide for use in producing fusion protein
LLELRSNFEAMKMEWQIS

SEQ ID NO: 68 - protein, 21 aa, Artificial Sequence
  Description of Artificial Sequence: Peptide for use in producing fusion protein
DEELNQIFEAMKMYPLVHVTK

SEQ ID NO: 69 - skipped

SEQ ID NO: 70 - protein, 21 aa, Artificial Sequence
  Description of Artificial Sequence: Peptide for use in producing fusion protein
SNLVSLLHSQKILWTDPQSFG

SEQ ID NO: 71 - protein, 21 aa, Artificial Sequence
  Description of Artificial Sequence: Peptide for use in producing fusion protein
LFLHDFLNAQKVELYPVTSSG

SEQ ID NO: 72 - protein, 16 aa, Artificial Sequence
  Description of Artificial Sequence: Peptide for use in producing fusion protein
SDINALLSTQKIYWAH

SEQ ID NO: 73 - protein, 23 aa, Artificial Sequence
  Description of Artificial Sequence: Peptide for use in producing fusion protein
MASSLRQILDSQKMEWRSNAGGS

SEQ ID NO: 74 - skipped

SEQ ID NO: 75 - protein, 23 aa, Artificial Sequence
  Description of Artificial Sequence: Peptide for use in producing fusion protein
MAHSLVPIFDAQKIEWRDPFGGS

SEQ ID NO: 76 - protein, 23 aa, Artificial Sequence
  Description of Artificial Sequence: Peptide for use in producing fusion protein
MGPDLVNIFEAQKIEWHPLTGGS

SEQ ID NO: 77 - protein, 23 aa, Artificial Sequence
  Description of Artificial Sequence: Peptide for use in producing fusion protein
MAFSLRSILEAQKMELRNTPGGS

SEQ ID NO: 78 - protein, 23 aa, Artificial Sequence
  Description of Artificial Sequence: Peptide for use in producing fusion protein
MAGGLNDIFEAQKIEWHEDTGGS

SEQ ID NO: 79 - protein, 23 aa, Artificial Sequence
  Description of Artificial Sequence: Peptide for use in producing fusion protein
MSSYLAPIFEAQKIEWHSAYGGS

SEQ ID NO: 80 - protein, 23 aa, Artificial Sequence
  Description of Artificial Sequence: Peptide for use in producing fusion protein
MAKALQKILEAQKMEWRSHPGGS

SEQ ID NO: 81 - protein, 23 aa, Artificial Sequence
  Description of Artificial Sequence: Peptide for use in producing fusion protein
MAFQLCKIFYAQKMEWHGVGGGS

SEQ ID NO: 82 - protein, 23 aa, Artificial Sequence
  Description of Artificial Sequence: Peptide for use in producing fusion protein
MAGSLSTIFDAQKIEWHVGKGGS

SEQ ID NO: 83 - protein, 23 aa, Artificial Sequence
  Description of Artificial Sequence: Peptide for use in producing fusion protein
MAQQLPDIFDAQKIEWRIAGGGS

SEQ ID NO: 84 - protein, 23 aa, Artificial Sequence
  Description of Artificial Sequence: Peptide for use in producing fusion protein
MAQRLFHILDAQKIEWHGPKGGS

SEQ ID NO: 85 - protein, 23 aa, Artificial Sequence
  Description of Artificial Sequence: Peptide for use in producing fusion protein
MAGCLGPIFEAQKMEWRHFVGGS

SEQ ID NO: 86 - protein, 23 aa, Artificial Sequence
  Description of Artificial Sequence: Peptide for use in producing fusion protein
MAWSLKPIFDAQKIEWHSPGGGS

SEQ ID NO: 87 - protein, 23 aa, Artificial Sequence
  Description of Artificial Sequence: Peptide for use in producing fusion protein
MALGLTRILDAQKIEWHRDSGGS

SEQ ID NO: 88 - protein, 23 aa, Artificial Sequence
  Description of Artificial Sequence: Peptide for use in producing fusion protein
MAGSLRQILDAQKIEWRRPLGGS

SEQ ID NO: 89 - protein, 23 aa, Artificial Sequence
  Description of Artificial Sequence: Peptide for use in producing fusion protein
MADRLAYILEAQKMEWHPHKGGS